(19) United States
(12) Patent Application Publication
(10) Pub. No.: US 2023/0025985 A1
(43) Pub. Date: Jan. 26, 2023

(54) LARGE VESSEL OCCLUSION EARLY NOTIFICATION AND EMERGENCY MEDICAL ROUTING

(71) Applicant: Open Water Internet Inc., San Francisco, CA (US)

(72) Inventors: Achal Singh Achrol, Highland, CA (US); Soren Konecky, Alameda, CA (US)

(21) Appl. No.: 17/872,652

(22) Filed: Jul. 25, 2022

Related U.S. Application Data
(60) Provisional application No. 63/225,671, filed on Jul. 26, 2021.

(51) Int. Cl. A61B 5/00; A61B 5/11; A61B 5/02; G01C 21/36

(52) U.S. Cl. CPC A61B 5/746; A61B 5/7275; A61B 5/1112; A61B 5/0013; A61B 5/0022; A61B 5/02007; G01C 21/3626; A61B 5/0082

(57) ABSTRACT

A large vessel occlusion (LVO) alert generated by a point-of-care diagnostic system is received. The LVO alert is representative of a probability of an LVO event in a patient. A mapping coordinate is received. A database of care centers proximate to the mapping coordinate is accessed. A preferred care center from the database is selected based at least on a treatment capability associated with the preferred care center and a distance of the care centers to the mapping coordinate. An LVO event notification is transmitted to initiate stroke medical care.

### CROSS-REFERENCE TO RELATED APPLICATIONS

This application claims priority to U.S. provisional Application No. 63/225,671 filed Jul. 26, 2021, which is hereby incorporated by reference. This application is related to a U.S. non-provisional patent application entitled, “Large Vessel Occlusion Alert from Optical Measurements,” filed the same day.

### TECHNICAL FIELD

This disclosure relates generally to medical devices, and in particular to large vessel occlusion (LVO) detection.

### BACKGROUND INFORMATION

Disruption of blood flow to any part of the brain is a medical emergency. When blood flow is interrupted due to a blockage of the large vessels supplying blood to the brain, the normal function of the brain becomes impaired resulting in a stroke, with associated symptoms of impaired consciousness, speech, numbness, weakness, and/or paralysis of parts of the body. Early detection and treatment of stroke can reduce the permanent damage to the portions of the brain where blood supply was disrupted.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified.

FIG. 1 illustrates a system for generating an LVO alert and transmitting an LVO event notification to a medical care center, in accordance with aspects of the disclosure.

FIG. 2 illustrates example memory that includes data associated with example care centers, in accordance with aspects of the disclosure.

FIG. 3 illustrates an example interface for routing an ambulance to a preferred care center, in accordance with aspects of the disclosure.

FIG. 4 illustrates an example process of transmitting an LVO event notification to a preferred care center, in accordance with aspects of the disclosure.

FIG. 5 illustrates an example process of early notification of LVO, in accordance with aspects of the disclosure.

FIGS. 6A-6B illustrates example optical devices initiating optical measurements of the right hemisphere of the brain and the left hemisphere of the brain, in accordance with aspects of the disclosure.

FIG. 6C illustrates an example block diagram schematic for capturing optical readings in an optical device, in accordance with aspects of the disclosure.

FIG. 7 illustrates an example device for placing on a head, in accordance with aspects of the disclosure.

FIGS. 8A-8C illustrates a bottom view (standard axial imaging orientation) of a head including various regions of the head related to LVO detection, in accordance with aspects of the disclosure.

FIG. 9 illustrates an example positioning of an optical device with respect to various regions of a brain, in accordance with aspects of the disclosure.

FIG. 10 illustrates an approximate measurement position for optical imaging relative to a skull, in accordance with aspects of the disclosure.

FIG. 11 illustrates various optical imaging regions with respect to a skull, in accordance with aspects of the disclosure.

FIG. 12 illustrates a side view of an example placement of an optical device on a head of a patient, in accordance with aspects of the disclosure.

FIG. 13 illustrates a front view of example placements of optical devices on a head of a patient, in accordance with aspects of the disclosure.

FIGS. 14A-14B illustrate an example headset for placing on a head of a patient, in accordance with aspects of the disclosure.

FIGS. 15A-15E illustrates optical readings from the right and left hemisphere of a brain for generating an LVO alert, in accordance with aspects of the disclosure.

FIG. 16 illustrates a device for securing to a head that includes a compression unit to selectively compress the right and left superficial temporal artery (STA) of a patient for optical readings, in accordance with aspects of the disclosure.

FIG. 17 illustrates a device for securing to a head that includes a compression unit to selectively compress the right and left STA of a patient for optical readings, in accordance with aspects of the disclosure.

FIG. 18 illustrates an example process of generating an LVO alert, in accordance with aspects of the disclosure.

FIG. 19 illustrates an example process of generating an LVO alert in response to optical measurement, in accordance with aspects of the disclosure.

### BRIEF SUMMARY OF THE INVENTION

A device for early notification of large vessel occlusion (LVO) includes an alert input, a wireless radio, a global positioning system (GPS) chip, and processing logic. The alert input is configured to receive an LVO alert generated by a point-of-care diagnostic system. The GPS chip is configured to generate a mapping coordinate of the device. The processing logic is coupled to the alert input, the wireless radio, and the GPS chip. The processing logic is configured to receive the LVO alert from the alert input, receive a mapping coordinate from the GPS chip, and access a database of care centers proximate to the mapping coordinate. The LVO alert may be representative of a probability of an LVO event in a patient. The processing logic is further configured to select a preferred care center from the database based at least on: (1) a treatment capability associated with the preferred care center; and (2) a distance of the care centers to the mapping coordinate. The processing logic transmits, with the wireless radio, an LVO event notification to the preferred care center to initiate stroke medical care.

In an implementation, the processing logic is further configured to receive a cerebrovascular image associated with the LVO alert. Transmitting the LVO event notification to the preferred care center with the wireless radio may include transmitting the cerebrovascular image.

In an implementation, the device further includes a memory storing the database of care centers and the processing logic is communicatively coupled to read the memory to access the database.

In an implementation, accessing the database of care centers includes transmitting an access request to a database via the wireless radio and receiving, in response to the access request, a database response that includes a plurality of care centers and resources and locations of the care centers. The access request may include the mapping coordinates. The database response may be received via the wireless radio.

In an implementation, the LVO alert is received by the wireless radio and the wireless radio is configured to interface with at least one of a cellular network, a wireless local area network (WLAN), or a satellite communication system.

In an implementation, the LVO alert includes an indicator of a subtype of LVO. The subtype of LVO may be an ICA occlusion, an MCA occlusion, an M2 inferior occlusion, or an M2 superior occlusion, for example.

In an implementation of the disclosure, a computer-implemented method includes receiving a large vessel occlusion (LVO) alert generated by a point-of-care diagnostic system, receiving a mapping coordinate, and accessing a database of care centers proximate to the mapping coordinate. The LVO alert may be representative of a probability of an LVO event in a patient. The computer-implemented method further includes selecting a preferred care center from the database based at least on: (1) a treatment capability associated with the preferred care center; and (2) a distance of the care centers to the mapping coordinate. An LVO event notification is transmitted to the preferred care center to initiate stroke medical care.

In an implementation, routing instructions to the preferred care center are determined based on the mapping coordinate and a location of the preferred care center and the routing instructions to the preferred care center are presented to a user interface.

In an implementation, a cerebrovascular image associated with the LVO alert is received and transmitting the LVO event notification to the preferred care center includes transmitting the cerebrovascular image. A cerebrovascular distribution consistent with an LVO may be highlighted in the cerebrovascular image.

In an implementation, the computer-implemented method further includes driving a display to present an identification of the preferred care center.

In an implementation, the mapping coordinate is a GPS coordinate.

In an implementation, the database includes a plurality of care centers and the care centers in the database have medical resources associated with the care centers. The care centers in the database may have a location or address of the care center.

In an implementation, the computer-implemented method further includes determining the distance to the plurality of care centers by calculating a current distance based on the mapping coordinate and the location or address of the care centers.

In an implementation, the computer-implemented method further includes transmitting an access request to a database via a cellular network and receiving, in response to the access request, a database response that includes a plurality of care centers and resources and locations of the care centers. The access request may include the mapping coordinate. The database response may be received via the cellular network.

In an implementation, selecting the preferred care center from the database is also based on a travel-time to the preferred care center.

In an implementation, the computer-implemented method further includes receiving a subsequent mapping coordinate and transmitting the subsequent mapping coordinate to the preferred care center.

A In an implementation, the LVO alert includes an indicator of a subtype of LVO. The subtype of LVO may be an ICA occlusion, an MCA occlusion, an M2 inferior occlusion, or an M2 superior occlusion, for example.

In an example method of the disclosure, a first optical measurement of tissue with a first optical device is initiated. The first optical measurement includes a first shallow optical reading and a first deeper optical reading. The first optical device may be placed to measure a right hemisphere of a brain. A second optical measurement of tissue with a second optical device is initiated. The second optical measurement includes a second shallow optical reading and a second deeper optical reading. The first optical measurement and the second optical measurement may be executed during a shared (same) time period. The second optical device may be placed to measure a left hemisphere of the brain. A first difference value between the first shallow optical reading and the first deeper optical reading is determined. A second difference value between the second shallow optical reading and the second deeper optical reading is determined. A ratio of the first difference value to the second difference value is calculated. If the ratio is greater than the threshold value, an LVO alert is generated. The method may be a computer-implemented method.

In an implementation, the first difference value is a first Area Under the Curve (AUC) between the first shallow optical reading and the first deeper optical reading and the second difference value is a second AUC between the second shallow optical reading and the second deeper optical reading. If the first difference value is a first number and the second difference value is a second number that is three times larger than a first number, it may indicate asymmetric blood flow between the right hemisphere and the left hemisphere. Hence, an LVO alert may be generated.

In an implementation, the method further includes driving a compression unit configured to temporarily occlude blood flow in a superficial temporal artery (STA). The STA is temporarily occluded during the first optical measurement and the second optical measurement.

In an implementation, the first optical measurement is directed to measure left blood flow relating to a left internal carotid artery (ICA) and left middle cerebral artery (MCA) and the second optical measurement is directed to measure right blood flow relating to a right ICA and a right MCA.

In an implementation, the first shallow optical reading is measured by a first photodetector of the first optical device that is spaced between 5 mm and 15 mm from a light source of the first optical device and the first deeper optical reading is measured by a second photodetector of the first optical device that is spaced between 30 mm to 45 mm from the light source of the first optical device. The second shallow optical reading may be measured by a third photodetector of the second optical device that is spaced between 5 mm and 15 mm from a light source of the second optical device and the first deeper optical reading may be measured by a fourth photodetector of the second optical device that is spaced between 30 mm to 45 mm from the light source of the second optical device.

In an implementation, the first optical measurement includes a first middle optical reading having a depth between the first shallow optical reading and the first deeper optical reading. The second optical measurement includes a second middle optical reading having the depth of the first middle optical reading. In this implementation, generating the LVO alert is also in response to the first middle optical reading and the second middle optical reading. The LVO alert may be generated in response to additional optical readings.

In an implementation of the disclosure, a device for placing on a head includes a first optical device, a second optical device, and processing logic. The first optical device is configured to measure a right hemisphere of a brain of a user of the device. The second optical device is configured to measure a left hemisphere of the brain of the user of the device. The processing logic is configured to initiate a first optical measurement of tissue with the first optical device and initiate a second optical measurement of the tissue with the second optical device. The first optical measurement includes a first shallow optical reading and a first deeper optical reading. The second optical measurement includes a second shallow optical reading and a second deeper optical reading. The processing logic is further configured to determine a first difference value between the first shallow optical reading and the first deeper optical reading, determine a second difference between the second shallow optical reading and the second deeper optical reading, and generate a large vessel occlusion (LVO) alert when a ratio of the first difference value to the second difference value is larger than a threshold value.

In an implementation the device further includes a compression unit configured to selectively compress a superficial temporal artery (STA) of the user. The processing logic may be configured to drive the compression unit to temporarily occlude the STA of the user during the first optical measurement and the second optical measurement. In an implementation, the compression unit includes a pneumatic pressure cuff sized to go around the head of the user. In an implementation, the device includes a frame for securing the device to the head of the user and the compression unit includes an actuator that applies direct pressure to an STA region of the head of the user. The actuator is supported by the frame of the device.

In an implementation, the first optical device is disposed on the device to perform the first optical measurement over a right medial forehead of the user to image a right anterior cerebral artery (ACA) region of the head and the second optical device is disposed on the device to perform the second optical measurement over a left medial forehead of the user to image a left ACA region of the head.

In an implementation, the first optical device is disposed on the device to perform the first optical measurement over a right lateral forehead of the user to image a right superior M2 region of the head and the second optical device is disposed on the device to perform the second optical measurement over a left lateral forehead of the user to image a left superior M2 region of the head.

In an implementation, the first optical device is disposed on the device to perform the first optical measurement over a right temple of the user to image a right inferior M2 region of the head and the second optical device is disposed on the device to perform the second optical measurement over a left temple of the user to image a left inferior M2 region of the head.

In an implementation, the first optical device is disposed on the device to perform the first optical measurement over a right temple of the user to image a right M1 region of the head and the second optical device is disposed on the device to perform the second optical measurement over a left temple of the user to image a left M1 region of the head.

In an implementation of the disclosure, a method includes occluding blood flow in a superficial temporal artery (STA), initiating a plurality of optical measurement while the blood flow in the STA is occluded, and generating a large vessel occlusion (LVO) alert in response to the plurality of optical measurements.

In an implementation, the plurality of optical measurements includes directing infrared light into a head of a user and measuring infrared exit signals that exit the head of the user. The infrared exit signals are portions of the infrared light scattered by the head of the user.

In an implementation, occluding blood flow in the STA includes driving a compression unit to selectively compress the STA.

### DETAILED DESCRIPTION

Embodiments of Large Vessel Occlusion (LVO) alert systems and methods of generating LVO alerts with optical devices are described herein. In the following description, numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In aspects of this disclosure, visible light may be defined as having a wavelength range of approximately 380 nm-700 nm. Non-visible light may be defined as light having wavelengths that are outside the visible light range, such as ultraviolet light and infrared light. Infrared light having a wavelength range of approximately 700 nm-1 mm includes near-infrared light. In aspects of this disclosure, near-infrared light may be defined as having a wavelength range of approximately 700 nm-1.6 μm.

The internal carotid arteries (ICAs) supply nutrients and oxygen to significant portions of the brain, including the frontal and temporal lobes, as well as deep central structures like the basal ganglia. The ICAs branch into the anterior cerebral arteries (ACAs) and the middle cerebral arteries (MCAs). Disruption of blood flow to the brain may be treated using a variety of medical treatments. Medical treatment with intravenous thrombolytics is limited to a small subset of patients who qualify and are within the appropriate time window, usually within 4.5 hours of symptom onset. Large vessel occlusions (LVOs) are resistant to medical therapy alone. While LVOs are responsible for approximately one third of all acute ischemic strokes, they account for 90% of the mortality from stroke and severe neurological disability in survivors. Endovascular thrombectomy (removing blood clot(s) via a catheter-based intervention in advanced imaging suites aimed at restoring blood flow through the occluded blood vessel) has emerged as a safe and effective treatment for LVOs and can be done even in patients who may not qualify for intravenous thrombolytics (systemic therapy with a “clot-busting” drug or biologic, often via intravenous infusion, aimed at restoring blood flow through the occluded blood vessel medically), including in extended time windows out to 24 hours in appropriately selected patients. Clinical trials have shown that patients with LVOs in the ICA or MCA in whom blood flow could be restored via endovascular therapy have significantly better outcomes than patients who were managed only medically. In addition, time to endovascular treatment has been shown to be a critical factor in achieving good functional outcomes. Despite the overall finding that thrombectomy improves outcomes in LVO, the majority of patients in pooled analysis across all thrombectomy trials were nonetheless left with poor functional outcome of severe dependent morbidity or death. If patients are able to get to thrombectomy within 2.5 hours of symptom onset, however, over 90% of them achieve good functional outcome. A major problem is that patients with LVO are often routed initially to the closest hospital, which creates significant inter-facility transfer delays once an LVO is diagnosed to transport the patient to a thrombectomy-capable hospital. In addition, pre-hospital notification of the fact that an LVO patient is being sent to the receiving facility has shown to reduce time to intervention by 40 minutes. Therefore, there is a significant need for a rapid point-of-care solution that can detect LVO in the field to improve time to endovascular treatment through early notification and directly routing these patients to the closest endovascular therapy center.

Human tissue is translucent to infrared light, although different parts of the human body (e.g. skin, blood, bone) exhibit different absorption and scattering coefficients. In implementations of the disclosure, Large Vessel Occlusion (LVO) alerts are generated using optical imaging of a person's head. In particular, the optical imaging of the blood flow in the internal carotid artery (ICA) and middle cerebral artery (MCA) on each hemisphere (left and right) of the brain may be used to generate an LVO alert. Detecting a LVO (indicative of a need for acute ischemic stroke emergency care activation and coordination) earlier in medical care is important to limit brain damage and save lives. Additionally, detecting an LVO and also providing the relevant medical care from a care facility that has the necessary facilities, medical equipment (e.g. endovascular capable center), and physicians currently available on-call to address an LVO will significantly enhance patient outcomes. Implementations of the systems and methods described in this disclosure may relate to the analysis of cerebral blood flow (CBF) in the brain using near-infrared laser intensity and speckle contrast analyses for the purpose of point-of-care LVO detection, automated early notification, and improved routing decisions by emergency medical service (EMS).

In implementations of the disclosure, the point-of-care optical imaging may be carried out by an imaging system configured to emit laser light through a first optical fiber into a tissue sample (e.g. head), detect diffused light returning from the tissue sample through a second optical fiber, capture an image of the diffused light carrying inherent characteristics (e.g. features) of the underlying tissue, and determine blood flow data within the tissue sample at least partially based on features of coherent light interference patterns and intensities in the image. The imaging system may emit laser light using one or more coherent light sources having one or more optical fibers coupled to one or more coherent light sources. The imaging system may detect diffused light using one or more light detectors having one or more optical fibers coupled to one or more image sensors. The imaging system may determine blood characteristics based on the image by using processing logic coupled to the light sources and light detectors. The devices, systems, and/or techniques described in U.S. non-provisional patent application Ser. No. 16/904,572 may be used in some of the implementations of the disclosure, for example. U.S. non-provisional patent application Ser. No. 16/904,572 filed Jun. 18, 2020 is expressly incorporated by reference herein.

In illustrative implementations, measurements of the blood flow in the brain of an individual can be obtained at the point-of-care using near-infrared laser light transmitted through the scalp via one or multiple optical fiber(s) in order to propagate photons down through the underlying brain tissue. A second optical fiber, which may be a multimode optical fiber, receives the exit signal of the laser light exiting the sample, which is imaged by a camera sensor. Multiple such cameras can be configured to receive exit signals via separate optical fibers positioned in such a way as to receive the respective exit signals from different depths of interrogation. Characteristics of the light intensity measured by the average pixel values of the image can be related to the optical properties of the underlying brain tissue, such as concentration of hemoglobin (cerebral blood volume) and/or oxygenation of hemoglobin (cerebral oximetry). Additionally, when photons in the laser light encounter moving objects such as cerebral blood flow, it results in a doppler shift effect that broadens the spectrum and reduces the coherence of the laser light. Less coherence corresponds to increased blood flow, and more coherence corresponds to decreased blood flow. The coherence of the exit signals determined from the images captured by one or more cameras is processed using an image processing technique to calculate the standard deviation over the mean of the image. This image processing technique is a way of expressing the coherence as a measurement of laser speckle contrast (cerebral blood flow).

In an example implementation, the data obtained from the example above-described point-of-care laser system can be used to perform measurements of cerebral blood flow (CBF), cerebral blood volume (CBV) and/or cerebral oximetry or oxygen saturation (SO2) in patients suspected of having a stroke. For visualization purposes, the measurements associated with a given tissue property can be indicated by a color scale value in a representative image. For example, measurements of relatively higher light intensity average pixel values can be displayed as higher color scale values for CBV and SO2 (e.g. green), versus lower light intensity average pixel values having lower color scale values (e.g. red). Similarly, measurements of less coherence in speckle contrast can be displayed as higher color scale values for CBF (e.g. green), versus more coherence displayed as decreased blood flow (e.g. red).

As described in the implementation herein, differences between the above parameters of CBF, CBV and/or SO2 located within each hemisphere of the brain can be compared. For example, measurements made on the forehead and temple scalp on the left hemisphere corresponding to regions of blood flow relating to the left internal carotid artery (ICA) and left middle cerebral artery (MCA), can be compared to measurements made on the forehead and temple scalp on the right hemisphere, corresponding to regions of blood flow relating to the right ICA and right MCA. The extent of the differences between symmetrically located vascular distributions in the different hemispheres of the brain can indicate a probability that there is a large vessel occlusion (LVO) of an associated underlying blood vessel segment. In situations where the differences between the measurements of symmetrically located vascular distributions in different hemispheres of the brain are greater than a threshold difference, the probability that there is an LVO of an underlying blood vessel segment can be determined as greater than a threshold probability. As a result, a point-of-care detection of LVO can be performed and a resulting early notification can be provided to one or more clinicians for expedited review to result in more timely treatment decisions, including decisions on how to triage and route the patient differently, e.g. to transport directly to a thrombectomy-capable center with early notification to prepare the catheter lab ahead of time for the patient. In one or more implementations, automated computer algorithms using the output of implementations described herein as their input can trigger these alerts, communications, and routing decisions automatically.

In general terms, the blood flow between the right hemisphere of the brain and the left hemisphere of the brain is usually equal and therefore have a ratio of approximately one (plus or minus noise). However, if there is an underlying large vessel occlusion (due to loss of blood flow-related signal on one side) the ratio is different (e.g. outside the normal range) due to the loss of signal on one side. In an implementation, a ratio between the right and left blood flow being less than 0.8 indicates an LVO. In an implementation, a ratio between the right and left blood flow being more than 1.2 indicates an LVO. In an implementation, a ratio between the right and left blood flow being less than 0.6 indicates an LVO. In an implementation, a ratio between the right and left blood flow being more than 1.4 indicates an LVO.

Alternatively, in one or more additional implementations, the presence of an LVO can be determined without a comparison between different hemispheres of the brain. In such examples, the CBF, CBV, and/or SO2 values for a number of locations can be compared directly to one or more known threshold values and normal ranges. In situations where the measured values at one or more locations within the brain of the patient are less than a threshold value, an LVO may be detected.

In some implementations of the disclosure, the superficial temporal artery (STA), is temporarily occluded (e.g. by manual compression against the scalp with a pneumatic pressure cuff, direct compression, or other similar technique) during certain optical measurements in order to remove the noise (blood flow) signal generated by the STA that is not representative of blood flow in the ICA and MCA (the target of the blood flow measurements). The STA may be temporarily occluded by using a compression unit that is mounted on or about the head. The compression unit may gently (temporarily) push up against the STA to temporarily occlude the STA.

The implementations described herein can identify large vessel occlusions (LVOs) of the brain in an automated manner at the point-of-care. The abnormalities can be numerically represented or visually depicted in images that can be provided to clinicians, as well as automatically trigger notifications and re-routing decisions. In this way, the amount of time between onset of an LVO stroke, diagnosis by a clinician, and definitive treatment with endovascular therapy can be minimized, which can in turn maximize the potential for good functional outcomes.

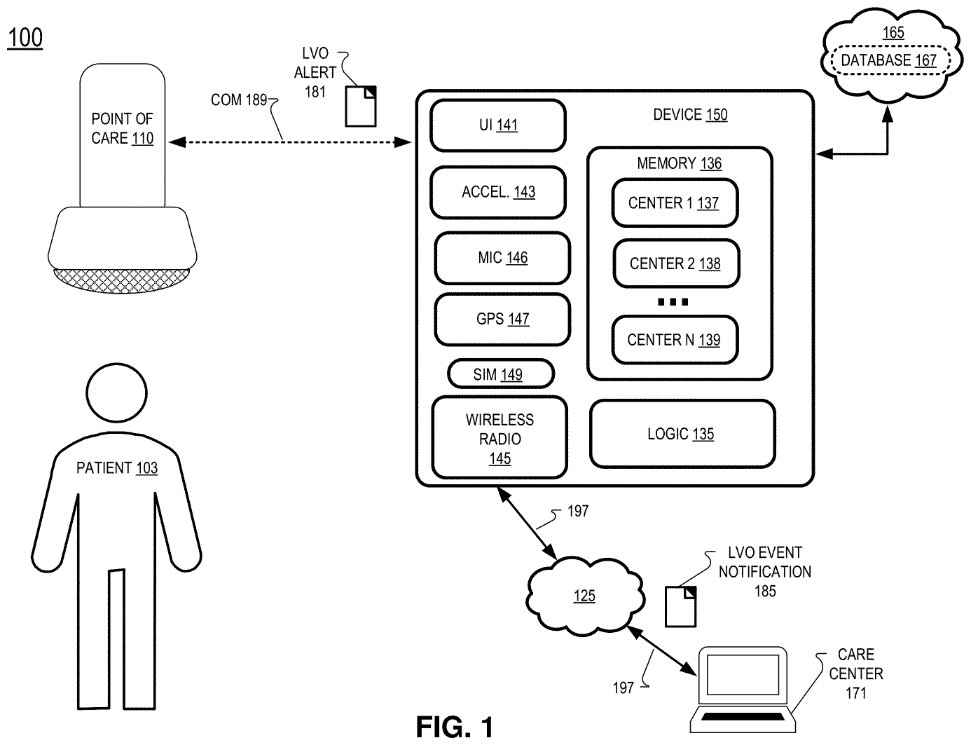

FIG. 1 illustrates a system 100 for generating an LVO alert and transmitting an LVO event notification to a medical care center, in accordance with implementations of the disclosure. System 100 includes a point of care diagnostic system 110. Point of care diagnostic system 110 may also be referred to as a device 110 in this disclosure. Device 110 may include optical imaging hardware. Device 110 may be shaped as a wand to image tissue (e.g. head) of patient 103 or may be implemented as a head mounted medical device that is sized to be fitted onto a head of a patient 103. Based on imaging by device 110, device 110 may generate an LVO alert 181 representative of a probability of an LVO event (potentially leading to an LVO stroke) in patient 103. In some implementations, the LVO alert 181 is generated in response to a blood flow difference between the right hemisphere and left hemisphere of the brain of patient 103. LVO alert 181 may be an analog or digital message that is transmitted to device 150 over communication channel 189 (which may be wired or wireless).

In the illustration of FIG. 1, device 150 includes processing logic 135, memory 136, wireless radio 145 (e.g. IEEE 802.11 and/or cellular radios). The illustrated example device 150 also includes an accelerometer 143, a microphone 146, and a global positioning sensor (GPS) 147. Device 150 may include various user interface (UI) elements 141. For example, device 150 may include a touchscreen interface and/or buttons. Device 150 may be a mobile device such as a smartphone or tablet. Device 150 may be a laptop or desktop computer. Device 150 may be travelling with an ambulance or other mobile care unit and thus the location of device 150 is a proxy for a location of the patient 103 that is being imaged by point of care diagnostic system 110. Device 150 may be connected to a network 165 via wired or wireless communications. Network 165 may be a remote network or be considered “the cloud” by those skilled in the art.

In the illustrated implementation of FIG. 1, memory 136 includes data corresponding to medical care centers. In FIG. 1, memory 136 includes data corresponding to n care centers where n is an integer number. Data about care centers may be loaded locally on device 150 or the data about care centers may be updated on device 150 based on a GPS coordinate (location) of device 150. For example, if device 150 is located in a metropolitan area that has multiple care centers (e.g. hospitals), device 150 may access database 167 in network 165 to load proximate care centers into local memory 136. In some implementations, the data corresponding to proximate care centers remains in network 165 and is not downloaded locally to memory 136. A proximate care center may be defined as a care center within a certain distance to the GPS coordinate of device 150. For example, proximate care centers may be care centers within 100 miles of device 150.

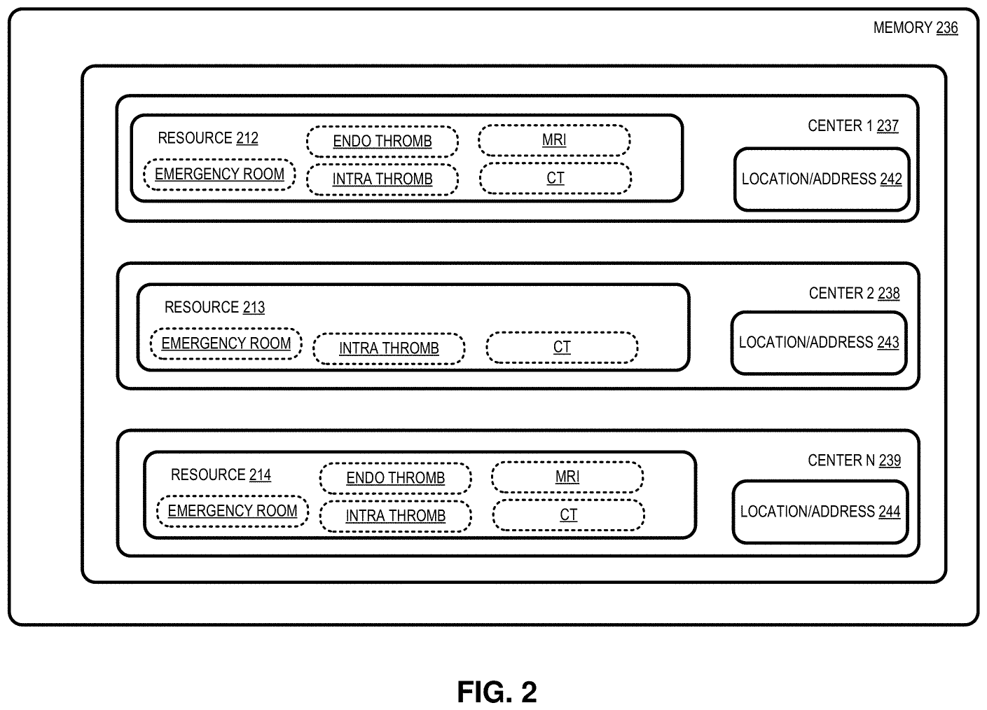

FIG. 2 illustrates example memory 236 that includes data associated with example care centers, in accordance with implementations of the disclosure. Memory 236 is an example that could be included in device 150 as memory 136. Care center 1 237 includes a location and/or address 242 of Care Center 1 and medical resources 212 that are associated with Care Center 1. Care Center 1 resources 212 include an emergency room and is equipped for endovascular thrombectomy and intravenous thrombolytics. Care Center 1 also includes an MRI and CT imaging resources. Care Center 2 238 includes a location and/or address 243 of Care Center 2 and medical resources 213 that are associated with Care Center 2. Care Center 2 resources 213 include an emergency room and is equipped for intravenous thrombolytics, but not endovascular thrombectomy. Care Center 2 has CT imaging but not MRI. Care Center n 239 includes a location and/or address 244 of Care Center n and medical resources 214 that are associated with Care Center n. Care Center n medical resources 214 include an emergency room and is equipped for endovascular thrombectomy and intravenous thrombolytics. Care Center n 239 also includes an MRI and CT imaging resources in resources 214. In some implementations, the data associated with the care center also includes whether on-call physicians are available at the hospital that are capable of a particular LVO therapy so that the care center could provide a specific LVO therapy in a given time frame.

In operation, device 150 may receive LVO alert 181 from device 110. LVO alert 181 may represent a probability that a Large Vessel Occlusion event has occurred in patient 103. LVO alert 181 may include a cerebrovascular image of patient 103. LVO alert 181 may include numerical data as to the probability of an LVO event. LVO alert 181 may include data associated with blood flow in particular areas of the brain, in some implementations. In an implementation, LVO alert 181 includes an indicator of a subtype of Large Vessel Occlusion that may have occurred. For example, the Large Vessel Occlusion may be an ICA occlusion, an MCA occlusion, an M2 inferior occlusion, or an M2 superior occlusion. For the purposes of this disclosure, a “Large Vessel Occlusion” may be defined as an occlusion of any vessel in the cranial area. Device 150 may then receive a mapping coordinate (e.g. a coordinate from GPS 147). In some implementations, point-of-care device 110 includes a GPS chip and provides the mapping coordinate. Device 150 may further access a database of care centers proximate to the mapping coordinate. The database of care centers may be stored locally in memory 136 or device 150 may access a cloud database 167 to access the care centers proximate to the mapping coordinate. Device 150 may further select a preferred care center based at least one of the following: (1) a treatment capability associated with the preferred care center; and (2) a distance of the care facilities (in the database) to the mapping coordinate. After selecting the preferred care center from the database, device 150 may transmit an LVO event notification 185 via communication channel 197 to the selected preferred care center 171 (or a device/network associated with the selected preferred care center 171).

LVO event notification 185 may alert the preferred care center 171 that a patient with an LVO event or LVO stroke is enroute to the preferred care center for medical treatment. LVO event notification 185 may be transmitted to the preferred care center 171 via an intervening network 125. In some implementations, LVO event notification 185 may include a cerebrovascular image of patient 103 so that physicians and other medical professionals can prepare the facility for arrival of patient 103 to the preferred care center. In some implementations, numerical values related to blood flow in particular areas of the brain are included in LVO event notification 185. In some implementations, an indicator of a subtype of Large Vessel Occlusion that may have occurred is included in LVO event notification 185. For example, the Large Vessel Occlusion may be an ICA occlusion, an MCA occlusion, an M2 inferior occlusion, or an M2 superior occlusion.

In some implementations, device 150 may continue to receive subsequent mapping coordinates and transmit the subsequent mapping coordinates to the selected preferred care center to update the preferred care center as to updated location and estimated-time-of-arrival of the patient 103 that is travelling with device 150 (e.g. in an ambulance that is carrying both device 150 and patient 103).

By way of example, selecting the preferred care center may be selected based on the closest care center (determined by the location/address of the care center compared to the mapping coordinate of device 150 or 110) that includes endovascular thrombectomy therapy resources. Hence, Care Center 2 238 in FIG. 2 may be 10 miles from device 150 but Care Center 1 may be selected as the preferred care center because it has the endovascular thrombectomy therapy resource, even though Care Center 1 is 12 miles from device 150 rather than 10 miles. In some implementations, the “closest” care center is determined based on a travel-time to the preferred care center. The travel-time may be determined from real-time mapping data that considers current traffic conditions, speed limits, and/or obstructions (e.g. lakes or mountains) that influence the travel-time to a particular care center. Where care centers are similarly equipped (e.g. Care Center n 239 and Care Center 1 237 each have endovascular thrombectomy therapy resources), the Care Center that is the closest or fastest to get to may be selected as the preferred care center. In some implementations, a current availability of a care center of providing a specific LVO therapy also factors into selecting the care center. The currently patient capacity of the care center and/or the availability of physicians that are available for performing the relevant LVO therapy may be factors for selecting the care center.

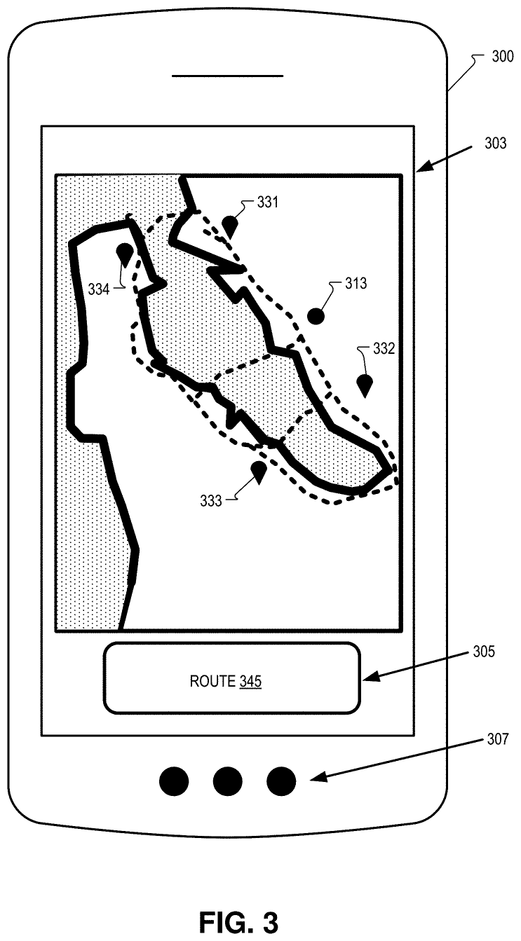

FIG. 3 illustrates an example interface for routing an ambulance to a preferred care center, in accordance with implementations of the disclosure. FIG. 3 illustrates an example device 300 that may be used as device 150, in some implementations. Device 300 may be a tablet or a smartphone. Device 300 includes a display 303 and physical/tactile user interface 307 (e.g. hardware buttons). A touchscreen overlaying display 303 may also allow for the user to interact with device 300 by way of software buttons such as software button 305.

An image of proximate care centers 331, 332, 333, and 334 are rendered to display 303 by processing logic (not illustrated) of device 300. Location 313 rendered to display 303 represents a current location of device 300 with respect to the care centers 331, 332, 333, and 334. After a preferred care center is selected by device 300 using one or more of the factors described above, routing guidance to the preferred care center may be rendered to display 303 to assist the ambulance to getting to the preferred care center.

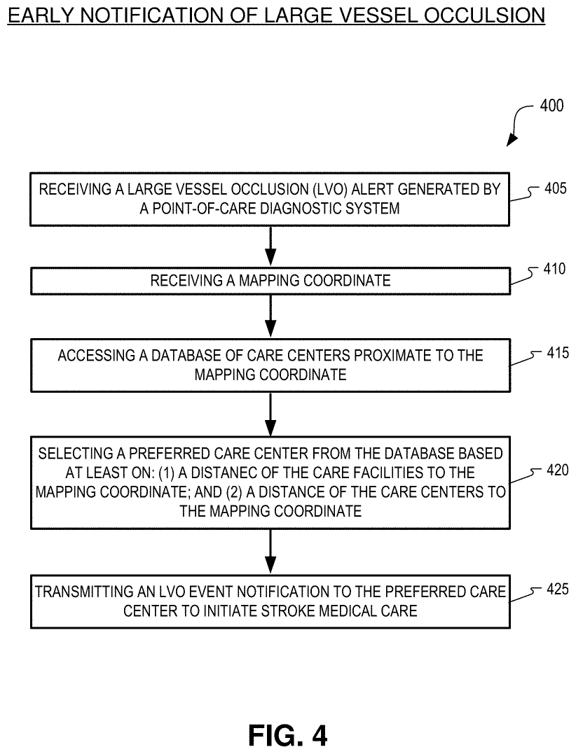

FIG. 4 illustrates an example process 400 of transmitting an LVO event notification to a preferred care center, in accordance with an implementations of the disclosure. The order in which some or all of the process blocks appear in process 400 should not be deemed limiting. Rather, one of ordinary skill in the art having the benefit of the present disclosure will understand that some of the process blocks may be executed in a variety of orders not illustrated, or even in parallel. Some or all of the process blocks in process 400 may be executed by a device 150. Some or all of the process blocks in process 400 may be executed by a remote server.

In process block 405, an LVO alert is received. The LVO alert (e.g. LVO alert 181) is generated by a point-of-care diagnostic system (e.g. system 110). The LVO alert is representative of a probability of an LVO event in a patient. The LVO alert includes an indicator of a subtype of LVO, in some implementations.

In process block 410, a mapping coordinate is received. The mapping coordinate may be a GPS coordinate. The mapping coordinate may be generated by a GPS chip on device 150 or device 110, for example.

In process block 415, a database of care centers is accessed. The care centers in the database are proximate to the mapping coordinate. The database may be stored locally on a device or the database may be located in a remote server in the cloud. The database may include a plurality of care centers. The care centers in the database may have medical resources associated with the care centers and the care centers in the database may have a location or address of the care center. Process 400 may further include determining the distance to the plurality of care centers by calculating a current distance based on the mapping coordinate and the location or address of the treatment centers.

In process block 420, a preferred care center is selected from the database based at least on: (1) a treatment capability associated with the preferred care center; and (2) a distance of the care centers to the mapping coordinate.

In process block 425, an LVO event notification (e.g. LVO event notification 185) is transmitted to the preferred care center to initiate stroke medical care at the preferred care center.

In an implementation, process 400 further includes: (1) determining routing instructions to the preferred care center based on the mapping coordinate and a location of the preferred care center; and (2) presenting the routing instructions to the preferred care center to a user interface.

In an implementation, process 400 further includes receiving a cerebrovascular image associated with the LVO alert. Transmitting the LVO event notification to the preferred care center may including transmitting the cerebrovascular image. In some implementations, a cerebrovascular distribution consistent with an LVO is highlighted in the cerebrovascular image.

In an implementation, process 400 further includes driving a display (e.g. display 303) to present an identification of the preferred care center.

In an implementation, process 400 further includes transmitting an access request to a database via a cellular network. The access request may include the mapping coordinate.

In an implementation, process 400 further includes transmitting an access request to a database via a cellular network and receiving, in response to the access request, a database response that includes a plurality of care centers and resources and locations of the care centers. The database response may be received via the cellular network and the access request may include the mapping coordinate.

In an implementation of process 400, selecting the preferred care center from the database is also based on a travel-time to the preferred care center.

In an implementation, process 400 further includes receiving a subsequent mapping coordinate and transmitting the subsequent mapping coordinate to the preferred care center.

In an implementation, the LVO alert includes an indicator of a subtype of LVO. The subtype of LVO may be an ICA occlusion, an MCA occlusion, an M2 inferior occlusion, or an M2 superior occlusion.

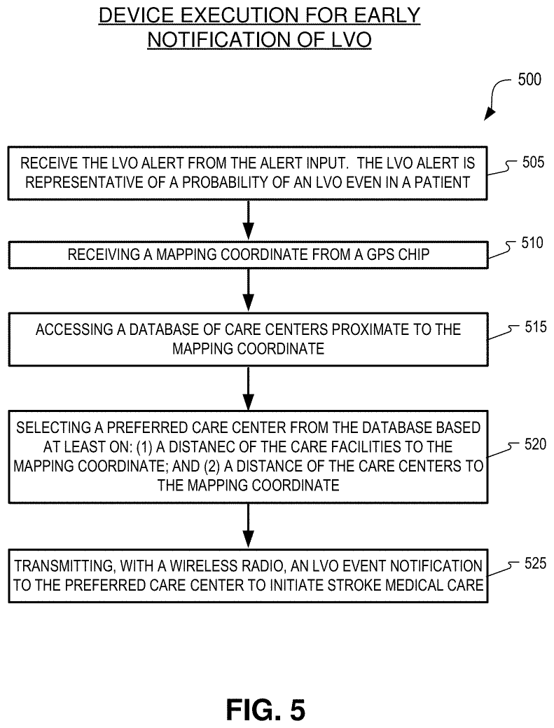

FIG. 5 illustrates an example process 500 of early notification of LVO, in accordance with an implementations of the disclosure. The order in which some or all of the process blocks appear in process 500 should not be deemed limiting. Rather, one of ordinary skill in the art having the benefit of the present disclosure will understand that some of the process blocks may be executed in a variety of orders not illustrated, or even in parallel. Some or all of the process blocks in process 500 may be executed by a device 150.

In process block 505, an LVO alert is received from an alert input of a device. The alert input may be a digital or analog input of the device. The alert input may be a wireless input received via a wireless radio. The LVO alert (e.g. LVO alert 181) is generated by a point-of-care diagnostic system (e.g. system 110). The LVO alert is representative of a probability of an LVO event in a patient. The LVO alert includes an indicator of a subtype of LVO, in some implementations.

In process block 510, a mapping coordinate is received from a GPS chip (e.g. element 147).

In process block 515, a database of care centers is accessed. The care centers in the database are proximate to the mapping coordinate. The database may be stored locally on a device. The database may include a plurality of care centers. The care centers in the database may have medical resources associated with the care centers and the care centers in the database may have a location or address of the care center. Process 500 may further include determining the distance to the plurality of care centers by calculating a current distance based on the mapping coordinate and the location or address of the treatment centers.

In process block 520, a preferred care center is selected from the database based at least on: (1) a treatment capability associated with the preferred care center; and (2) a distance of the care centers to the mapping coordinate.

In process block 525, an LVO event notification (e.g. LVO event notification 185) is transmitted to the preferred care center to initiate stroke medical care at the preferred care center.

In an implementation, process 500 further includes receiving a cerebrovascular image associated with the LVO alert. Transmitting the LVO event notification to the preferred care center may including transmitting the cerebrovascular image.

In an implementation of process 500, accessing the database of care centers includes transmitting an access request via the wireless radio and receiving a database response. The access request may include the mapping coordinates. The database response may include a plurality of plural care centers and resources and locations of the care center. The database response is received from the wireless radio.

In an implementation of process 500, the LVO alert is received by the wireless radio and the wireless radio is configured to interface with at least one of a cellular network, a wireless local area network (WLAN), or a satellite communication system.

In an implementation of process 500, the LVO alert includes an indicator of a subtype of LVO. The subtype of LVO may be an ICA occlusion, an MCA occlusion, an M2 inferior occlusion, or an M2 superior occlusion.

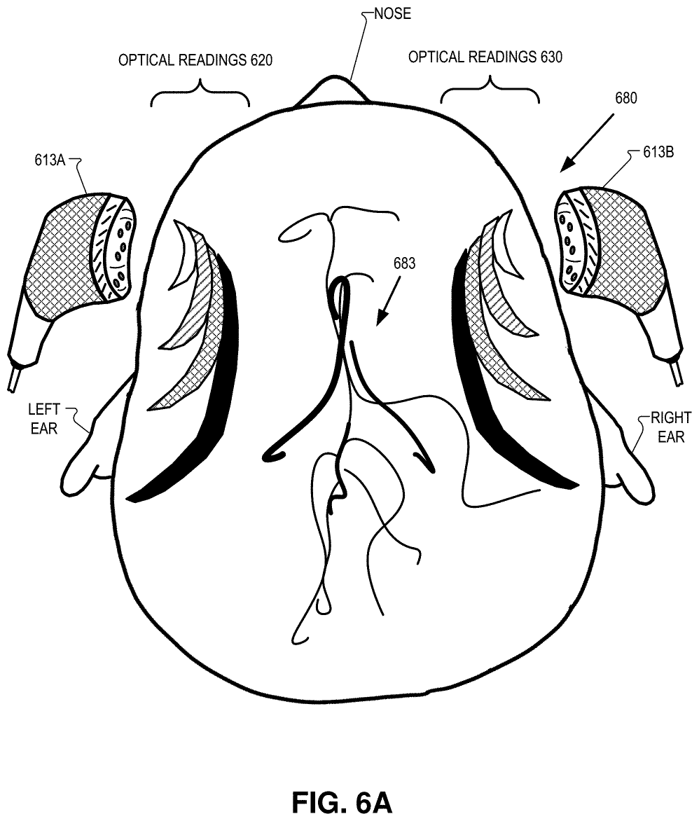

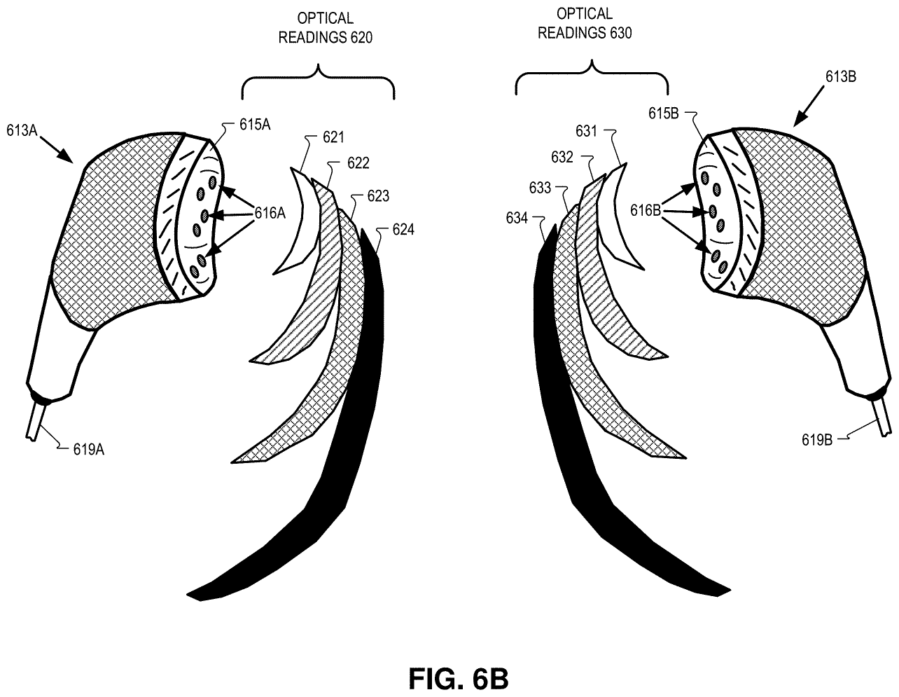

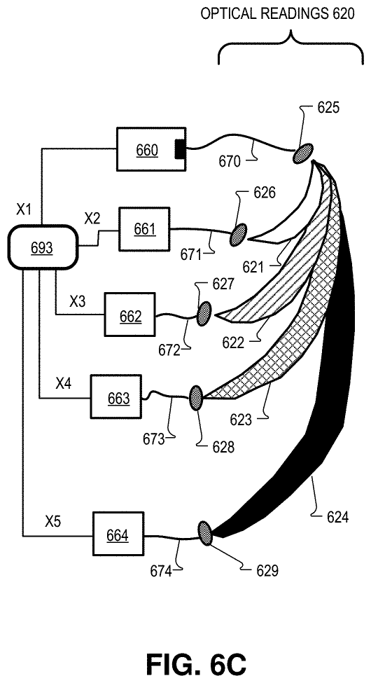

FIG. 6A illustrates example optical devices initiating optical measurements of the right hemisphere of the brain and the left hemisphere of the brain, in accordance with aspects of the disclosure. FIG. 6A is an overhead view of a head 680 including blood vessels and arteries 683. First optical device 613A is configured to measure a left hemisphere of the brain by way of optical readings 620. Second optical device 613B is configured to measure a right hemisphere of the brain by way of optical readings 630.

FIG. 6B illustrates a more detailed view of optical devices 613A and 613B, in accordance with aspects of the disclosure. Optical device 613A may be in the form factor of a “wand.” Optical device 613A may have a cord 619A that provides electrical power, laser light, and/or data lines to optical device 613A. Optical device 613A includes a face 615A that may come in contact with a head of a person for imaging purposes. Face 615A may have a curvature to assist in conforming to the head. Optical components 616A that facilitate optical readings 620 are disposed on or near face 615A. The optical components may include one or more fiber optic output ports to deliver infrared laser light into the brain, one or more fiber optic input ports to receive the infrared laser light exiting the brain, and/or image sensor(s) configured to capture images of infrared laser light exiting the brain.

FIG. 6B illustrates that optical readings 620 include optical readings 621, 622, 623, and 624. In other implementations, more or fewer optical readings may be measured by optical device 613A. Optical reading 621 is the shallowest optical reading, and optical reading 624 is the deepest optical reading. Optical reading 622 is deeper than optical reading 621 and optical reading 623 is deeper than optical reading 622. The depth of the optical readings may correspond with a distance of a detector (e.g. photodiode or image sensor) from an emitter or light source that provides the infrared laser light to the head/brain. In an implementation, optical reading 621 is measured by a detector that is 7.6 mm from the emitter/light source and is the shallowest optical reading. Optical reading 622 may be measured by a detector that is 14.5 mm from the emitter/light source and is the second shallowest optical reading. Optical reading 623 may be measured by a detector that is 36.1 mm from the emitter/light source and is the third shallowest optical reading. Optical reading 624 may be measured by a detector that is 42.8 mm from the emitter/light source and is the deepest optical reading. In some implementations, a detector may be 12 mm from the emitter/light source. In some implementations, a detector may be 36 mm from the emitter/light source. Of course, other depths are possible by adjusting the distance of the sensor (e.g. camera or photodiode) from the emitter or light source.

Optical device 613B may be similar to optical device 613A and may have a cord 619B that provides electrical power, laser light, and/or data lines to optical device 613A. Optical device 613B includes a face 615B that may come in contact with a head of a person for imaging purposes. Face 615B may have a curvature to assist in conforming to the head. Optical components 616B that facilitate optical readings 630 are disposed on or near face 615B. The optical components may include one or more fiber optic output ports to deliver infrared laser light into the brain, one or more fiber optic input ports to receive the infrared laser light exiting the brain, and/or image sensor(s) configured to capture images of infrared laser light exiting the brain.

Optical readings 630 include optical readings 631, 632, 633, and 634. In other implementations, more or fewer optical readings may be measured by optical device 613B. Optical reading 631 is the shallowest optical reading, and optical reading 634 is the deepest optical reading. Optical reading 632 is deeper than optical reading 631 and optical reading 633 is deeper than optical reading 632. The depth of the optical readings may correspond with a distance of a detector (e.g. photodiode or image sensor) from an emitter or light source that provides the infrared laser light to the head/brain. In an implementation, optical reading 631 is measured by a detector that is 7.6 mm from the emitter/light source and is the shallowest optical reading. Optical reading 632 may be measured by a detector that is 14.5 mm from the emitter/light source and is the second shallowest optical reading. Optical reading 633 may be measured by a detector that is 36.1 mm from the emitter/light source and is the third shallowest optical reading. Optical reading 634 may be measured by a detector that is 42.8 mm from the emitter/light source and is the deepest optical reading. In some implementations, a detector may be 12 mm from the emitter/light source. In some implementations, a detector may be 36 mm from the emitter/light source. Of course, other depths are possible by adjusting the distance of the sensor (e.g. camera or photodiode) from the emitter or light source.

FIG. 6C illustrates an example block diagram schematic for capturing optical readings in an optical device, in accordance with implementations of the disclosure. FIG. 6C includes processing logic 693 communicatively coupled to light source 660, photodetector 661, photodetector 662, photodetector 663, and photodetector 664, via communication channels X1, X2, X3, X4, and X5, respectively. Light source 660 may be an infrared light source. Light source 660 may be an infrared laser. In some implementations, a plurality of light sources may be included. The light sources may have different infrared wavelengths, in some implementations.

In the illustrated implementation, light source 660 provides illumination light to optical output port 625 by way of optical fiber 670. Processing logic 693 may drive light source 660 to selectively illuminate a head with illumination light. At least a portion of the illumination light may enter the head of a user. As discussed above, infrared light may propagate through tissue and eventually exit the tissue to be measured by photodetectors 661, 662, 663, and 664. Photodetectors 661, 662, 663, and 664 may include optical filters so that the photodetectors are sensitive to measure only the narrow-band wavelength emitted by light source 660 and block/reject other light wavelengths.

Example photodetectors 661, 662, 663, and 664 may be photodiodes or image sensors, for example. Processing logic 693 may initiate light measurements and/or image captures by photodetector 661. Photodetector 661 may receive a portion of the illumination light emitted by optical output port 625 as optical reading 621 that penetrates the tissue (e.g. head) at a first depth. Photodetector 661 may receive optical reading 621 by way of an input optical port 626 and optical fiber 671 that provides the light to photodetector 661.

Processing logic 693 may initiate light measurements and/or image captures by photodetector 662. Photodetector 662 may receive a portion of the illumination light emitted by optical output port 625 as optical reading 622 that penetrates the tissue (e.g. head) at a second depth. Photodetector 662 may receive optical reading 622 by way of an input optical port 627 and optical fiber 672 that provides the light to photodetector 662.

Processing logic 693 may initiate light measurements and/or image captures by photodetector 663. Photodetector 663 may receive a portion of the illumination light emitted by optical output port 625 as optical reading 623 that penetrates the tissue (e.g. head) at a third depth. Photodetector 663 may receive optical reading 623 by way of an input optical port 628 and optical fiber 673 that provides the light to photodetector 663.

Processing logic 693 may initiate light measurements and/or image captures by photodetector 664. Photodetector 664 may receive a portion of the illumination light emitted by optical output port 625 as optical reading 624 that penetrates the tissue (e.g. head) at a fourth depth. Photodetector 664 may receive optical reading 624 by way of an input optical port 629 and optical fiber 674 that provides the light to photodetector 664.

Input optical port 629 may be the farthest distance from output port 625, input optical port 628 may be the second farthest distance from output port 625, input optical port 627 may be the third farthest distance from output port 625, and input optical port 626 may be the closest to output port 625. While FIG. 6C illustrates an implementation that includes optical fibers and/or waveguides to provide and the receive light from the tissue, the light source(s) and photodetectors may be disposed close to (or even flush with) face 615A so that the light sources(s) provide the light directly to the tissue and the photodetectors receive the light directly—without including optical fibers to provide the light.

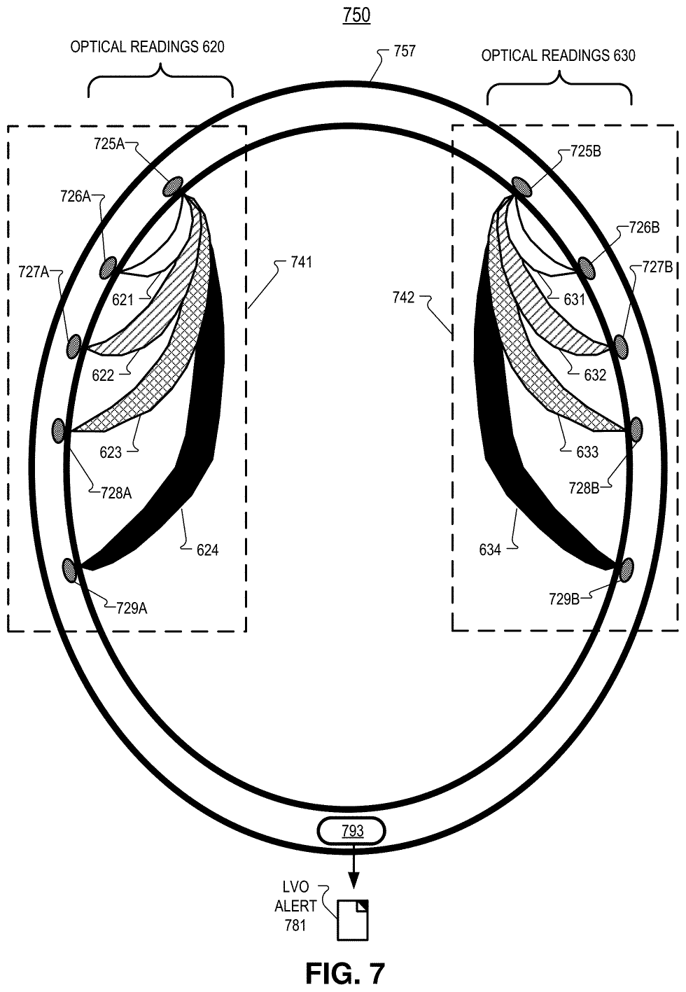

FIG. 7 illustrates an example device 750 for placing on a head, in accordance with implementations of the disclosure. Device 750 includes a frame 757 for securing device 750 to a head of a user. FIG. 7 illustrates that a first optical device 741 and a second optical device 742 may be incorporated in a single device 750 that may be secured to a head of a user for imaging purposes. First optical device 741 includes optical components 725A, 726A, 727A, 728A, 729A that may include the features of optical components 625, 626, 627, 628, 629, respectively. First optical device 741 may also be configured similarly to the schematic presented in FIG. 3C. Second optical device 742 includes optical components 725B, 726B, 727B, 728B, 729B that may include the features of optical components 625, 626, 627, 628, 629, respectively. Second optical device 742 may also be configured similarly to the schematic presented in FIG. 3C.

The illustrated example device 750 includes processing logic 793 configured to generate a LVO alert 781 based on optical measurements of tissue generated by first optical device 741 and second optical device 742. Processing logic 793 may be communicatively coupled to optical devices 741 and 742 to receive the optical measurements generated by optical devices 741 and 742.

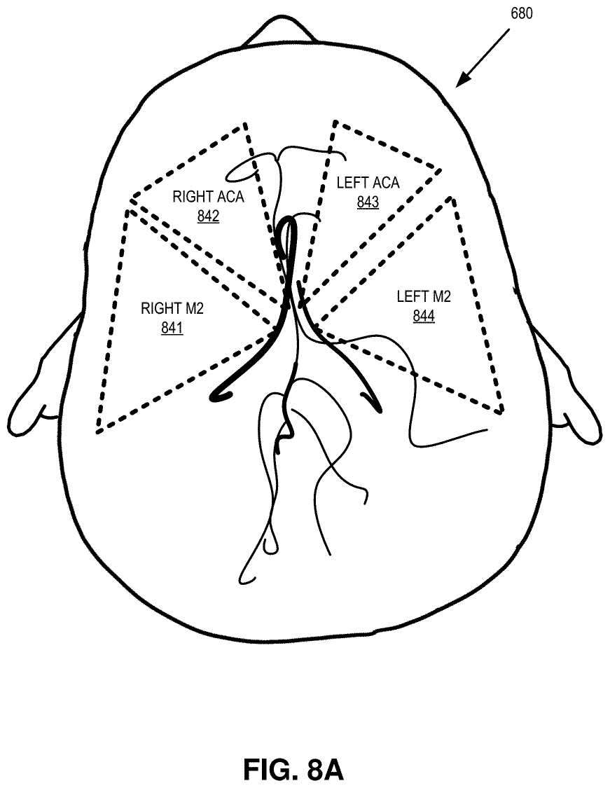

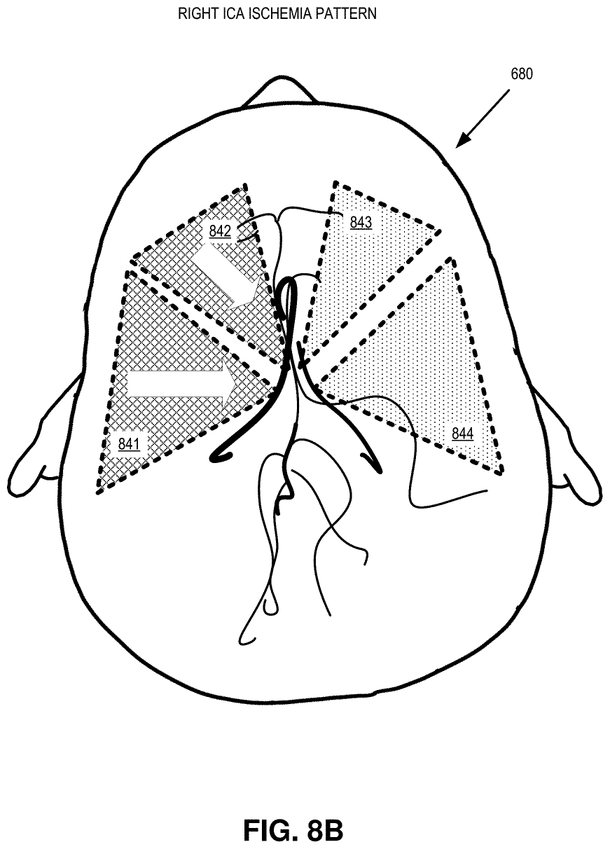

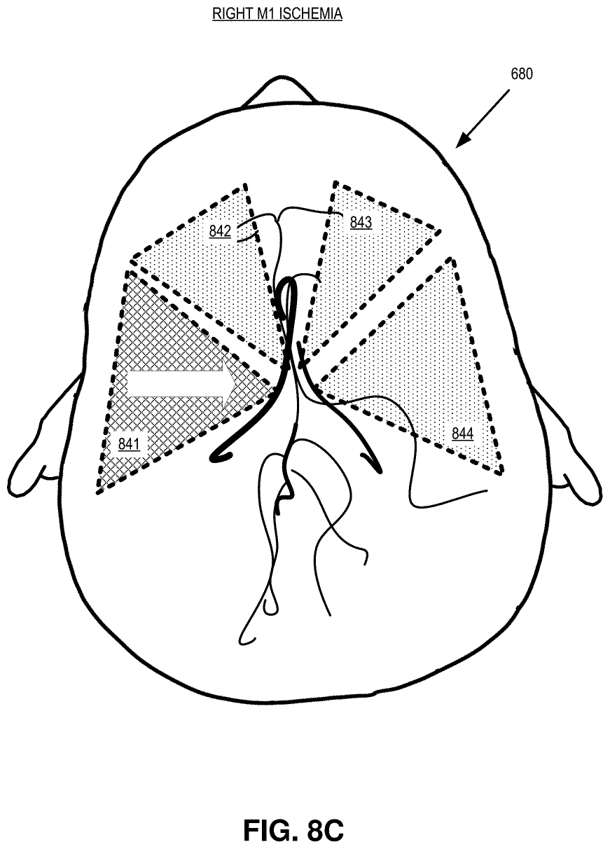

FIG. 8A illustrates a bottom view of head (standard axial imaging orientation) 680 including various regions of the head related to LVO detection, in accordance with implementations of the disclosure. FIG. 8A shows a right M2 region 841, a right ACA region 842, a left ACA region 843, and a left M2 region 844 of head 680. Right M2 region 841 may include a right lateral forehead over a right superior M2 region and a right temple over a right inferior M2 region of the head 680. Right ACA region 842 may be below a right medial forehead of head 680. Left ACA region 843 may be below a left medial forehead of head 680. Left M2 region 844 may include a left lateral forehead over a left superior M2 region and a left temple over a left inferior M2 region of the head 680.

FIG. 8B illustrates a right ICA ischemia pattern that may be measured by the optical devices, in accordance with implementations of the disclosure. In FIG. 8B, regions 843 and 844 on the left side of head 680 have normal blood flow while regions 841 and 842 on the right side of head 680 have abnormal blood flow consistent with an LVO of a right ICA ischemic stroke pattern (occlusion affecting right MCA+right ACA distribution). By performing optical measurements in accordance with this disclosure, the ischemic stroke pattern can be detected and an LVO alert may be generated to expedite medical treatment.

FIG. 8C illustrates a right M1 ischemia pattern that may be measured by the optical devices, in accordance with implementations of the disclosure. In FIG. 8C, regions, 842, 843, and 844 have normal blood flow while right M1 region 841 has abnormal blood flow consistent with an LVO of a right M1 ischemic stroke pattern (middle cerebral artery occluded). performing optical measurements in accordance with this disclosure, this ischemic stroke pattern can be detected and an LVO alert may be generated to expedite medical treatment.

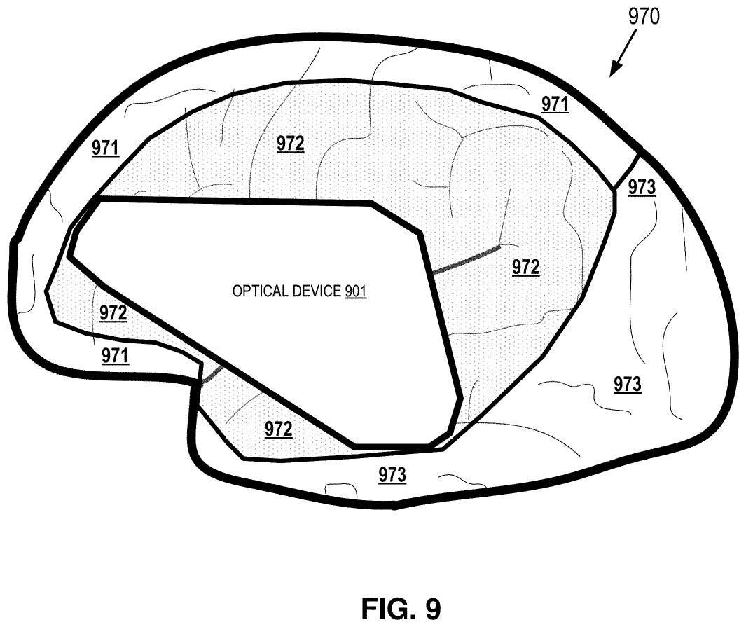

FIG. 9 illustrates an example positioning of an optical device 901 with respect to various regions of a brain 970, in accordance with implementations of the disclosure. FIG. 9 shows an ACA region 971, an MCA region 972, and a Posterior Cerebral Artery (PCA) region 973. Optical device 901 shows an example placement of an optical device over the MCA region 972 for optical imaging to detect LVOs.

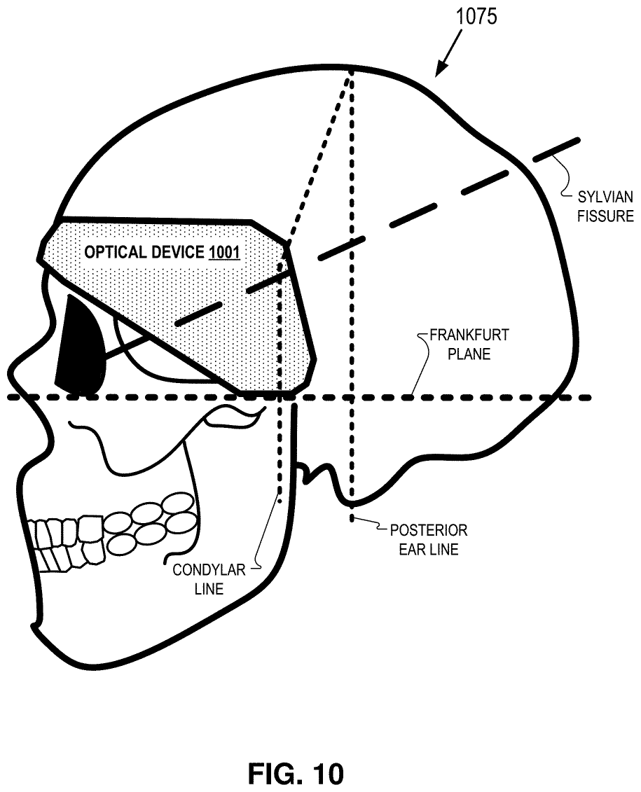

FIG. 10 illustrates an approximate measurement position for optical imaging relative to a skull 1075, in accordance with implementations of the disclosure. A Sylvian Fissure and a Frankfurt plane of skull 1075 are shown in FIG. 10. In addition, a Condylar line and a Posterior Ear Line are shown with respect to skull 1075. Optical device 1001 may be placed or fitted forward of the Posterior Ear Line for detecting of LVOs.

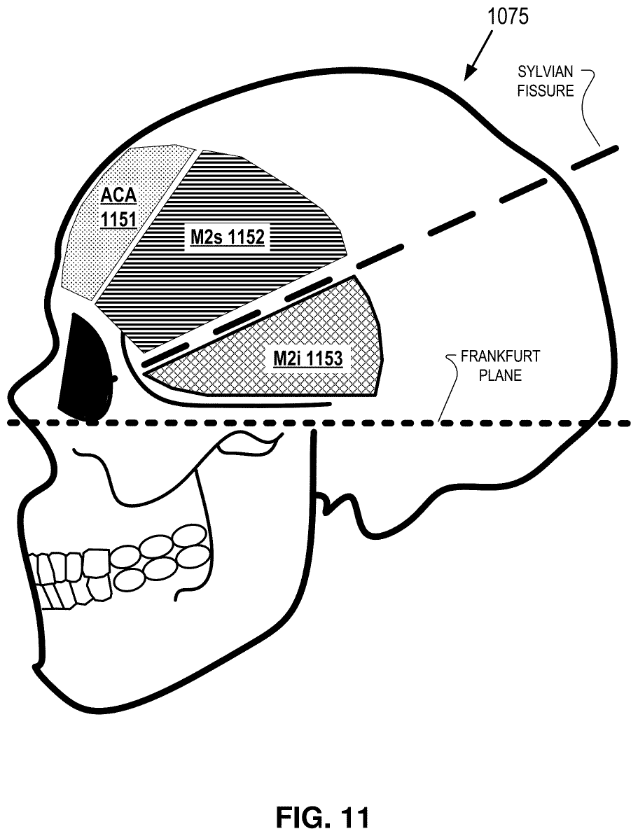

FIG. 11 illustrates various optical imaging regions with respect to skull 1075, in accordance with implementations of the disclosure. FIG. 11 shows an M2 inferior (M2ᵢ) region 1153 that is situated between the Sylvian Fissure and the Frankfurt plane of skull 1075. An M2 superior (M2ₛ) region 1152 is situated above the Sylvian Fissure and between ACA region 1151 and M2 inferior region 1153.

An optical device may be placed at different locations on the head to measure the different zones illustrated in FIGS. 8A-11. A lack of blood flow in a particular zone (as compared to the optical measurements of blood flow in the opposite hemisphere of the brain), may indicate a particular subtype of LVO. To facilitate this comparison, the position of the optical measurements may be symmetrical (e.g. optical measurement of right hemisphere positionally symmetric to optical measurement of left hemisphere) in specific regions corresponding to specific vascular territories. Example optical measurement locations are over the medial forehead for the anterior ACA, lateral forehead for the superior M2 division, over the temple for the inferior M2, and/or along the line depicted in FIG. 10 of the Sylvian fissure. In this way, a blood flow abnormality can be specific to what subtype of occlusion it is (e.g. ICA, MCA M1, M2 superior vs M2 inferior etc.).

If the optical measurements indicate a lack of blood flow in just the M2_{inferior} region 1153, LVO alert 181 may include an indicator that the LVO event is likely an M2 inferior occlusion. If the optical measurements indicate a lack of blood flow in just the M2ₛᵤₚₑᵣᵢₒᵣ region 1152, LVO alert 181 may include an indicator that the LVO event is likely an M2 superior occlusion. If the optical measurements indicate a lack of blood flow in the M2ₛᵤₚₑᵣᵢₒᵣ region 1152, the M2_{inferior} region 1153, and the ACA region 1151, LVO alert 181 may include an indicator that the LVO event is likely an ICA occlusion. If the optical measurements indicate a lack of blood flow in the M2ₛᵤₚₑᵣᵢₒᵣ region 1152 and the M2_{inferior} region 1153, LVO alert 181 may include an indicator that the LVO event is likely an MCA occlusion. In some implementations, the indicator of LVO subtype is included in LVO alert 181 and also in LVO event notification 185 to the care center.

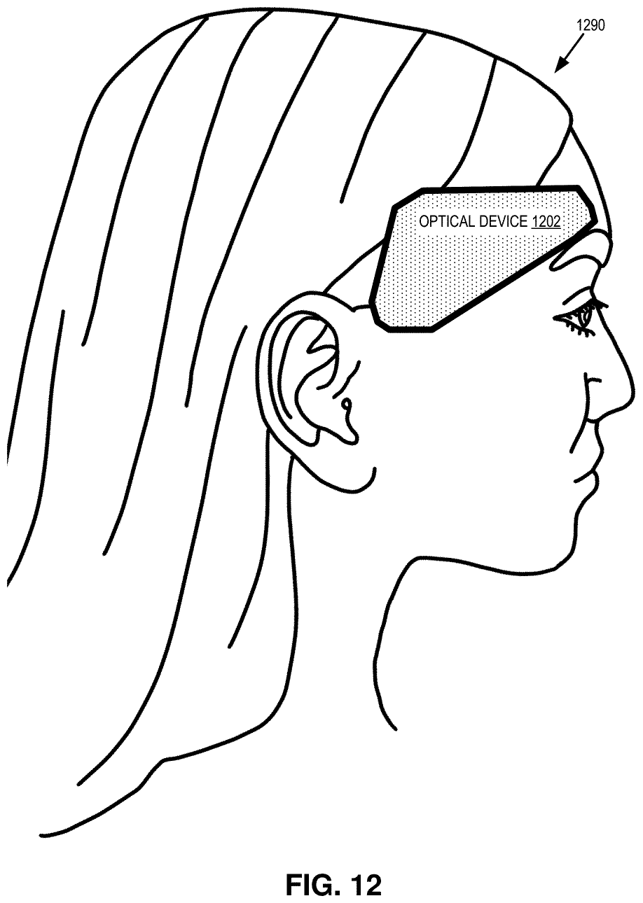

FIG. 12 illustrates a side view of an example placement of optical device 1202 on a head of patient 1290, in accordance with implementations of the disclosure. Optical device 1202 is positioned on the right side of the head over a right temple region of patient 1290, in the illustrated example of FIG. 12.

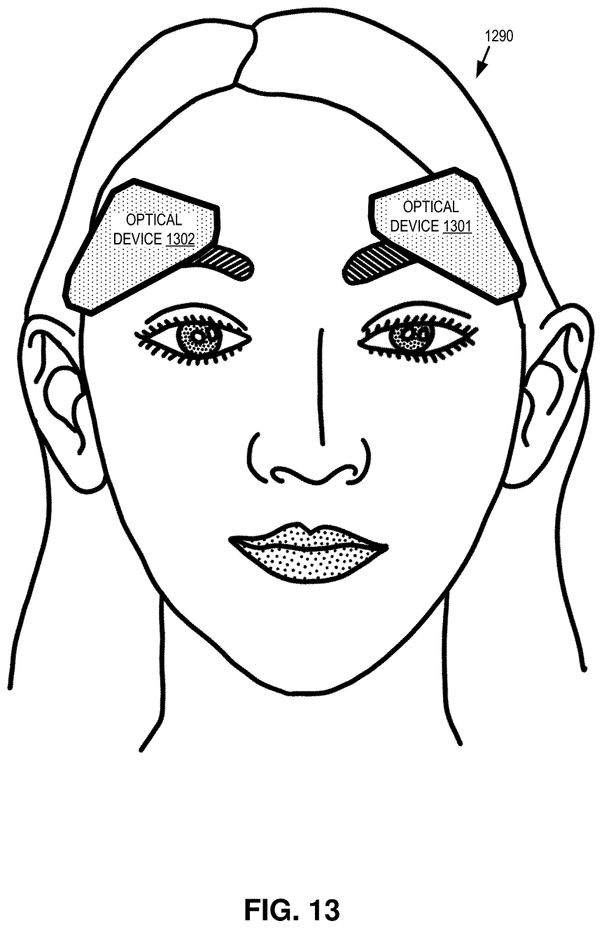

FIG. 13 illustrates a front view of example placements of optical devices 1301 and 1302 on a head of patient 1290, in accordance with implementations of the disclosure. Optical device 1302 is positioned on the right side of the head of patient 1290 and optical device 1301 is positioned on the left side of the head of patient 1290.

FIGS. 14A-14B illustrate an example headset 1499 for placing on a head of patient 1290, in accordance. Headset 1499 integrates optical devices 1401 and 1402 into headset 1499 so that optical devices 1401 and 1402 may be positioned to collect optical measurements of a left hemisphere and a right hemisphere of the brain during a same time period.

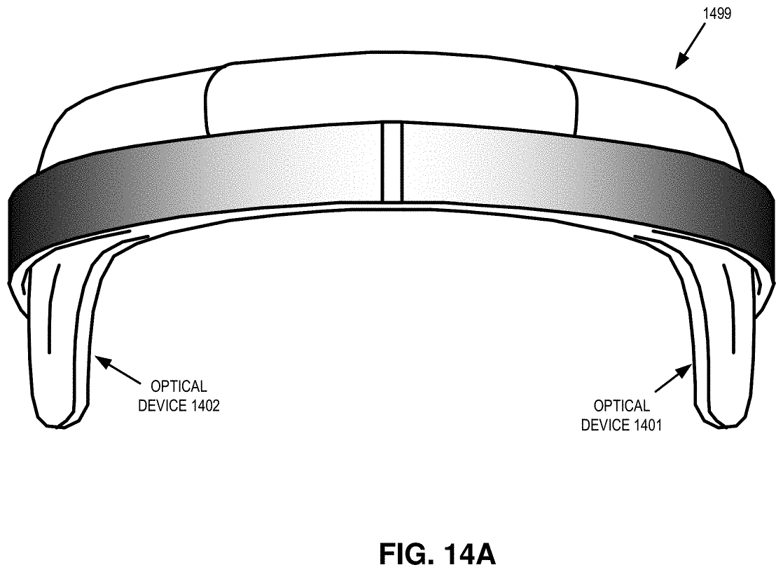

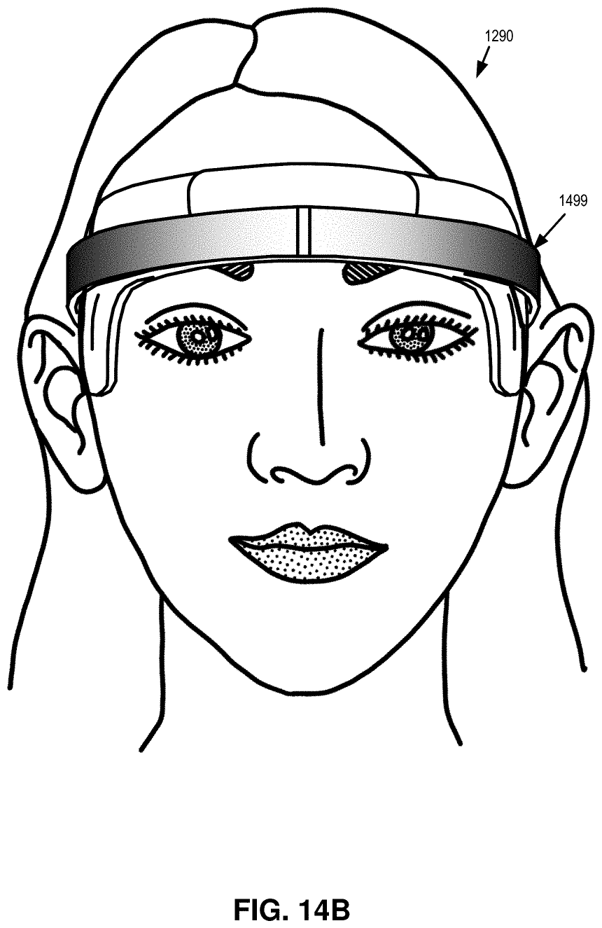

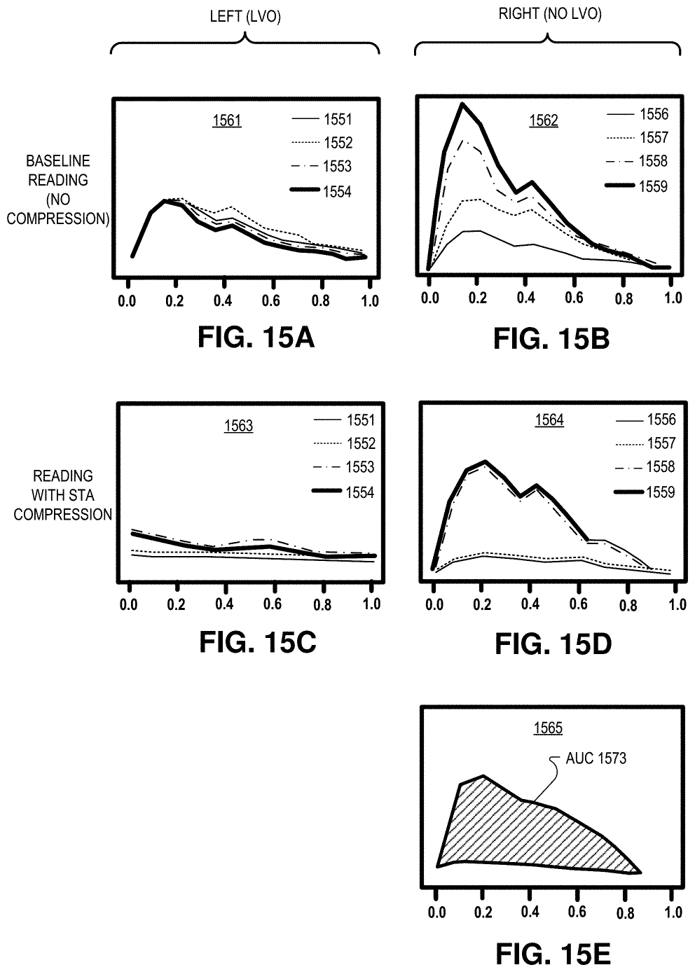

FIGS. 15A-15E illustrates baseline optical readings from the right and left hemisphere of a brain being compared to a threshold value for generating an LVO alert, in accordance with implementations of the disclosure. Chart 1561 of FIG. 15A illustrates a baseline reading of a left hemisphere of a brain where no compression is applied to a superficial temporal artery (STA). Solid trend line 1551 represents a shallowest optical reading (e.g. reading 621) from an optical device, dashed trend line 1552 represents a second shallowest optical reading (e.g. reading 622), dash-dot trend line 1553 represents a third shallowest optical reading (e.g. reading 623), and heavy-solid trend line 1554 represents the deepest optical reading (e.g. reading 624). Optical readings 1551, 1552, 1553, and 1554 are taken from a left side of the brain and may be generated by optical device 613A, 741, 1301, or 1401, for example. The x-axis of chart 1561 represents time in seconds and the y-axis of chart 1561 represents blood flow from a single pulse.

Chart 1562 of FIG. 15B illustrates a baseline reading of a right hemisphere of a brain where no compression is applied to the STA. Solid trend line 1556 represents a shallowest optical reading (e.g. reading 631) from an optical device, dashed trend line 1557 represents a second shallowest optical reading (e.g. reading 632), dash-dot trend line 1558 represents a third shallowest optical reading (e.g. reading 633), and heavy-solid trend line 1559 represents the deepest optical reading (e.g. reading 634). Optical readings 1556, 1557, 1558, and 1559 are taken from a right side of the brain and may be generated by optical device 613AB, 742, 1302, or 1402, for example. The x-axis of chart 1562 represents time in seconds and the y-axis of chart 1562 represents blood flow from a single pulse.

To reduce blood flow noise from the baseline readings, a compression unit may be activated to temporarily occlude blood flow in the STA for additional optical readings while the right and left STA is occluded by the compression unit. Temporarily occluding the STA may remove or reduce blood flow noise generated by the STA that manifest in the optical readings. Removing the blood flow noise generated by the STA may assist in isolating the blood flow signal generated by the ICA and MCA for a particular hemisphere of the brain.

Chart 1563 of FIG. 15C illustrates an STA compression reading of a left hemisphere of a brain where compression is applied to a left superficial temporal artery (STA). Solid trend line 1551 represents the shallowest optical reading (e.g. reading 621) from an optical device, dashed trend line 1552 represents the second shallowest optical reading (e.g. reading 622), dash-dot trend line 1553 represents the third shallowest optical reading (e.g. reading 623), and heavy-solid trend line 1554 represents the deepest optical reading (e.g. reading 624). The x-axis of chart 1563 represents time in seconds and the y-axis of chart 1563 represents blood flow from a single pulse.

Chart 1564 of FIG. 15D illustrates an STA compression reading of a right hemisphere of a brain where compression is applied to the right superficial temporal artery (STA). Solid trend line 1556 represents the shallowest optical reading (e.g. reading 631) from an optical device, dashed trend line 1557 represents the second shallowest optical reading (e.g. reading 632), dash-dot trend line 1558 represents the third shallowest optical reading (e.g. reading 633) and heavy-solid trend line 1559 represents the deepest optical reading (e.g. reading 634). The x-axis of chart 1564 represents time in seconds and the y-axis of chart 1564 represents blood flow from a single pulse. Using charts 1563 and 1564 (STA compression present) may assist in detecting an LVO since the variation of the optical readings due to scalp blood flow is reduced or eliminated.

Chart 1565 of FIG. 15E illustrates an Area Under the Curve (AUC) 1573 between the shallower optical readings of chart 1564 (optical readings 1556/1557) and the deeper optical readings of chart 1564 (optical readings 1558/1559). When the AUC is above a particular threshold, normal Cerebral Blood Flow (CBF) is indicated and no LVO is detected. Hence, AUC 1573 may indicate normal (healthy) blood flow in the right hemisphere of the brain. In contrast, the AUC difference between the shallower optical readings (optical readings 1551/1552) and the deeper optical readings of chart 1563 (optical readings 1553/1554) is quite small and may indicate abnormal blood flow of the left hemisphere of the brain. Hence, the AUC difference in chart 1563 may cause a device to generate an LVO alert (e.g. LVO alert 181 or 781). The smaller the AUC, the larger probability of an ischemia, in some implementations.

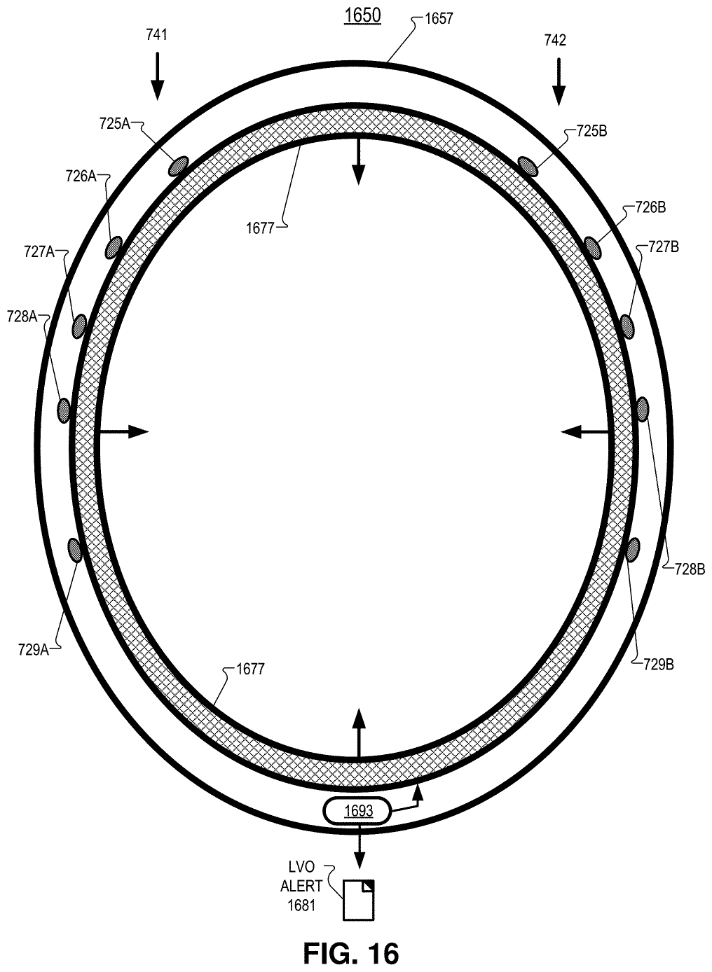

FIG. 16 illustrates a device 1650 for securing to a head that includes a compression unit to selectively compress the right and left STA of a patient for optical readings, in accordance with implementations of the disclosure. Device 1650 includes optical devices 741, 742, processing logic 1693, a frame 1657 and a pressure cuff 1677 that encircles the head of a patient. Pressure cuff 1677 is one example of a compression unit that can be used to selectively and temporarily occlude the right and left STA of a patient to generate the optical readings described in association with FIGS. 15A-15C. Processing logic 1693 may drive pressure cuff 1677 to inflate/expand in order to temporarily occlude the right and left STA of the patient. While the pressure cuff applies pressure to the right and left STA, the optical readings of FIGS. 15C and 15D can be generated. Pressure cuff 1677 may be a pneumatic pressure cuff. In some implementations, processing logic 1693 performs AUC calculations of the optical readings of FIGS. 15C and 15D to determine if the calculated AUCs are below a threshold value. If the AUC of either the right hemisphere or left hemisphere of the brain is below the threshold (indicating abnormal blood flow), processing logic 1693 may generate LVO alert 1681. LVO alert 1681 may be provided to device 150 of FIG. 1, for example.

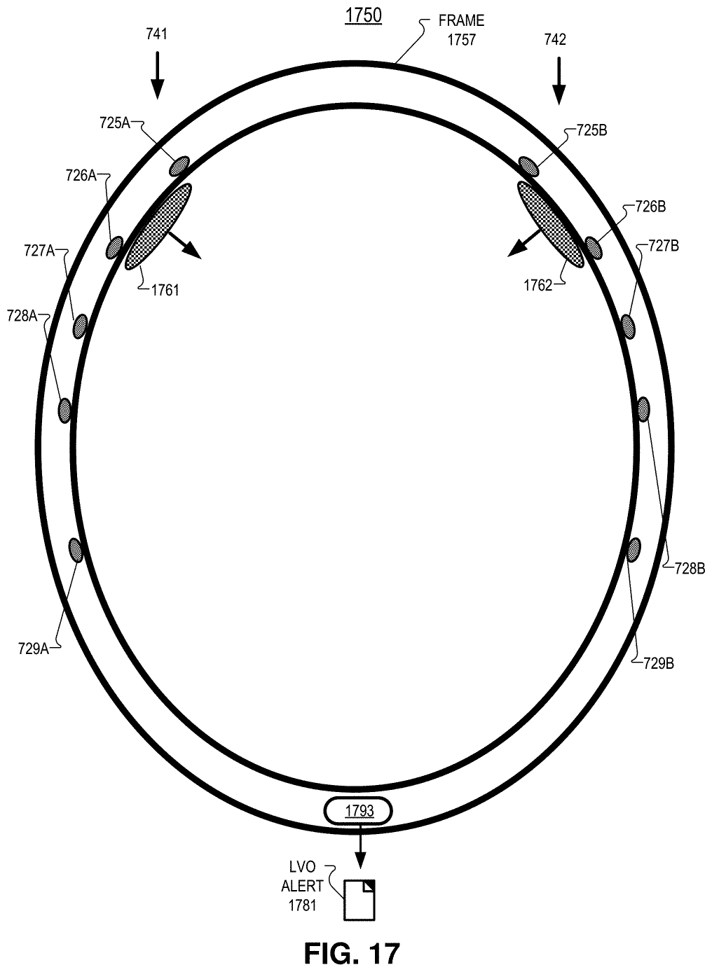

FIG. 17 illustrates a device 1750 for securing to a head that includes a compression unit to selectively compress the right and left STA of a patient for optical readings, in accordance with implementations of the disclosure. Device 1750 includes optical devices 741, 742, processing logic 1793, a frame 1757, and actuators 1761 and 1762. Actuators 1761 and 1762 are one example of a compression unit that can be used to selectively and temporarily occlude the right and left STA of a patient to generate the optical readings described in association with FIGS. 15A-15C. Processing logic 1793 may drive actuators 1761 and 1762 to apply direct pressure to an STA region of a head of a patient. The actuators 1761 and 1762 may be supported by frame 1757 in order to apply pressure to the STA region of the head to temporarily occlude the right and left STA of the patient. While the actuators apply pressure to the right and left STA, the optical readings of FIGS. 15C and 15D can be generated. In some implementations, processing logic 1793 performs AUC calculations of the optical readings of FIGS. 15C and 15D to determine if the calculated AUCs are below a threshold value. If the AUC of either the right hemisphere or left hemisphere of the brain is below the threshold (indicating abnormal blood flow), processing logic 1793 may generate LVO alert 1781. LVO alert 1781 may be provided to device 150 of FIG. 1, for example.

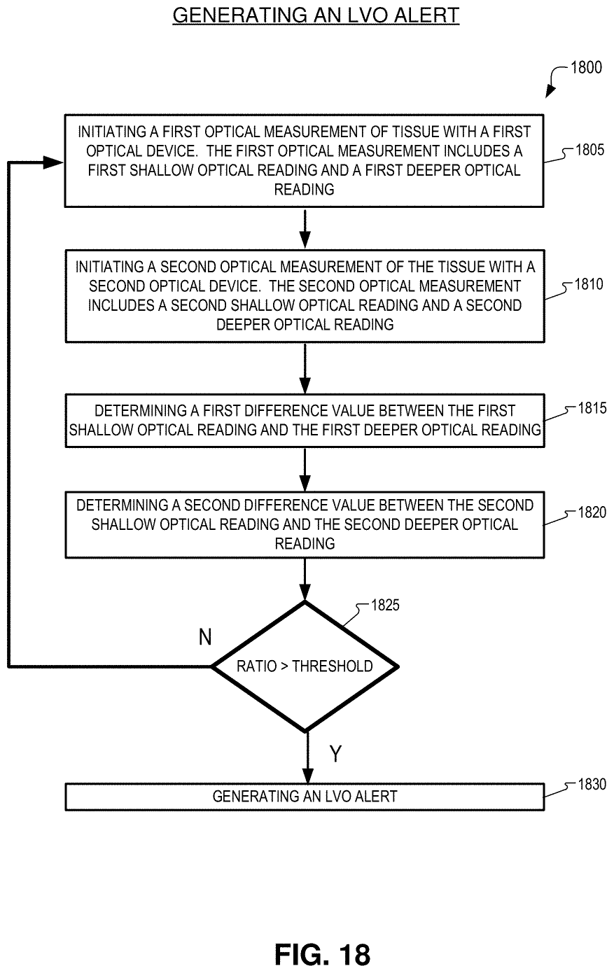

FIG. 18 illustrates an example process 1800 of generating an LVO alert, in accordance with an implementations of the disclosure. The order in which some or all of the process blocks appear in process 1800 should not be deemed limiting. Rather, one of ordinary skill in the art having the benefit of the present disclosure will understand that some of the process blocks may be executed in a variety of orders not illustrated, or even in parallel.

In process block 1805, a first optical measurement of tissue with a first optical device is initiated. The first optical measurement includes a first shallow optical reading and a first deeper optical reading. The first optical device may be placed to measure a right hemisphere of a brain.

In process block 1810, a second optical measurement of tissue with a second optical device is initiated. The second optical measurement includes a second shallow optical reading and a second deeper optical reading. The first optical measurement and the second optical measurement may be executed during a shared (same) time period. The second optical device may be placed to measure a left hemisphere of the brain.

In process block 1815, a first difference value between the first shallow optical reading and the first deeper optical reading is determined.

In process block 1820, a second difference value between the second shallow optical reading and the second deeper optical reading is determined.

In process block 1825, a ratio of the first difference value to the second difference value is calculated. If the ratio is less than a threshold value, process 1800 may return to process block 1805. If the ratio is greater than the threshold value, process 1800 may proceed to process block 1830 where an LVO alert is generated.

In an implementation of process 1800, the first difference value is a first Area Under the Curve (AUC) between the first shallow optical reading and the first deeper optical reading and the second difference value is a second AUC between the second shallow optical reading and the second deeper optical reading. If the first difference value is a first number and the second difference value is a second number that is three times larger than a first number, it may indicate asymmetric blood flow between the right hemisphere and the left hemisphere. Hence, an LVO alert may be generated.

Process 1800 may further include driving a compression unit configured to temporarily occlude blood flow in a superficial temporal artery (STA). The STA is temporarily occluded during the first optical measurement and the second optical measurement.

In an implementation of process 1800, the first optical measurement is directed to measure left blood flow relating to a left internal carotid artery (ICA) and left middle cerebral artery (MCA) and the second optical measurement is directed to measure right blood flow relating to a right ICA and a right MCA.

In an implementation of process 1800, the first shallow optical reading is measured by a first photodetector of the first optical device that is spaced between 5 mm and 15 mm from a light source of the first optical device and the first deeper optical reading is measured by a second photodetector of the first optical device that is spaced between 30 mm to 45 mm from the light source of the first optical device. The second shallow optical reading may be measured by a third photodetector of the second optical device that is spaced between 5 mm and 15 mm from a light source of the second optical device and the first deeper optical reading may be measured by a fourth photodetector of the second optical device that is spaced between 30 mm to 45 mm from the light source of the second optical device.

In an implementation of process 1800, the first optical measurement includes a first middle optical reading having a depth between the first shallow optical reading and the first deeper optical reading. The second optical measurement includes a second middle optical reading having the depth of the first middle optical reading. In this implementation, generating the LVO alert is also in response to the first middle optical reading and the second middle optical reading. The LVO alert may be generated in response to additional optical readings

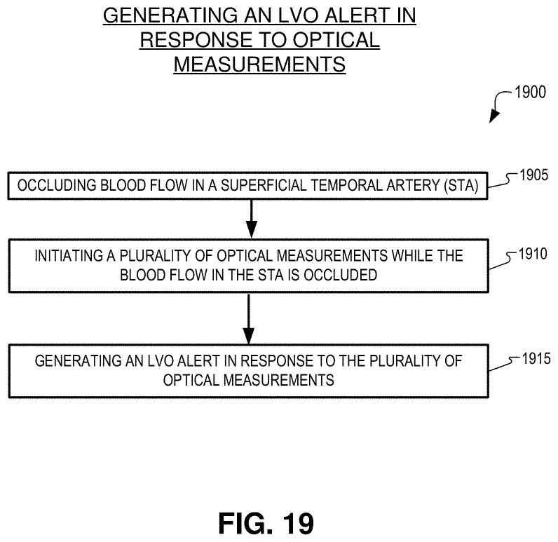

FIG. 19 illustrates an example process 1900 of generating an LVO alert in response to optical measurement, in accordance with an implementations of the disclosure. The order in which some or all of the process blocks appear in process 1900 should not be deemed limiting. Rather, one of ordinary skill in the art having the benefit of the present disclosure will understand that some of the process blocks may be executed in a variety of orders not illustrated, or even in parallel.

In process block 1905, blood flow is occluded in an STA.

In process block 1910, a plurality of optical measurements is initiated while the blood flow in the STA is occluded.

In process block 1915, an LVO alert is generated in response to the plurality of optical measurements.

In an implementation of process 1900, the plurality of optical measurements includes directing infrared light into a head of a user and measuring infrared exit signals that exit the head of the user. The infrared exit signals are portions of the infrared light scattered by the head of the user.

In an implementation of process 1900, occluding blood flow in the STA includes driving a compression unit to selectively compress the STA.

The term “processing logic” (e.g. processing logic) in this disclosure may include one or more processors, microprocessors, multi-core processors, Application-specific integrated circuits (ASIC), and/or Field Programmable Gate Arrays (FPGAs) to execute operations disclosed herein. In some embodiments, memories (not illustrated) are integrated into the processing logic to store instructions to execute operations and/or store data. Processing logic may also include analog or digital circuitry to perform the operations in accordance with embodiments of the disclosure.

A “memory” or “memories” described in this disclosure may include one or more volatile or non-volatile memory architectures. The “memory” or “memories” may be removable and non-removable media implemented in any method or technology for storage of information such as computer-readable instructions, data structures, program modules, or other data. Example memory technologies may include RAM, ROM, EEPROM, flash memory, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

Communication channels may include or be routed through one or more wired or wireless communication utilizing IEEE 802.11 protocols, BlueTooth, SPI (Serial Peripheral Interface), I²C (Inter-Integrated Circuit), USB (Universal Serial Port), CAN (Controller Area Network), cellular data protocols (e.g. 3G, 4G, LTE, 5G), optical communication networks, Internet Service Providers (ISPs), a peer-to-peer network, a Local Area Network (LAN), a Wide Area Network (WAN), a public network (e.g. “the Internet”), a private network, a satellite network, or otherwise.

A computing device may include a desktop computer, a laptop computer, a tablet, a phablet, a smartphone, a feature phone, a smartwatch, a server computer, or otherwise. A server computer may be located remotely in a data center or be stored locally.

The processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

The tangible non-transitory machine-readable storage medium includes any mechanism that provides (i.e., stores) information in a form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A computer-implemented method comprising:
receiving a large vessel occlusion (LVO) alert generated by a point-of-care diagnostic system, wherein the LVO alert is representative of a probability of an LVO event in a patient;
receiving a mapping coordinate;
accessing a database of care centers proximate to the mapping coordinate;
selecting a preferred care center from the database based at least on: (1) a treatment capability associated with the preferred care center; and (2) a distance of the care centers to the mapping coordinate; and
transmitting an LVO event notification to the preferred care center to initiate stroke medical care.

2. The computer-implemented method of claim 1 further comprising:
determining routing instructions to the preferred care center based on the mapping coordinate and a location of the preferred care center;
presenting the routing instructions to the preferred care center to a user interface.

3. The computer-implemented method of claim 1 further comprising:
receiving a cerebrovascular image associated with the LVO alert, wherein transmitting the LVO event notification to the preferred care center includes transmitting the cerebrovascular image.

4. The computer-implemented method of claim 3, wherein a cerebrovascular distribution consistent with an LVO is highlighted in the cerebrovascular image.

5. The computer-implemented method of claim 1 further comprising:
driving a display to present an identification of the preferred care center.

6. The computer-implemented method of claim 1, wherein the mapping coordinate is a GPS coordinate.

7. The computer-implemented method of claim 1, wherein the database includes a plurality of care centers, and wherein the care centers in the database have medical resources associated with the care centers, and wherein the care centers in the database have a location or address of the care center.

8. The computer-implemented method of claim 7 further comprising:
determining the distance to the plurality of care centers by calculating a current distance based on the mapping coordinate and the location or address of the care centers.

9. The computer-implemented method of claim 1 further comprising:
transmitting an access request to a database via a cellular network, wherein the access request includes the mapping coordinate; and
receiving, in response to the access request, a database response that includes a plurality of care centers and resources and locations of the care centers, wherein the database response is received via the cellular network.

10. The computer-implemented method of claim 1, wherein selecting the preferred care center from the database is also based on a travel-time to the preferred care center.

11. The computer-implemented method of claim 1 further comprising:
receiving a subsequent mapping coordinate; and
transmitting the subsequent mapping coordinate to the preferred care center.

12. The computer-implemented method of claim 1, wherein the LVO alert includes an indicator of a subtype of LVO.

13. The computer-implemented method of claim 12, wherein the subtype of LVO is an ICA occlusion, an MCA occlusion, an M2 inferior occlusion, or an M2 superior occlusion.

14. A device for early notification of large vessel occlusion (LVO) comprising:
an alert input configured to receive a large vessel occlusion (LVO) alert generated by a point-of-care diagnostic system;
a wireless radio;
a global positioning system (GPS) chip configured to generate a mapping coordinate of the device; and
processing logic coupled to the alert input, the wireless radio, and the GPS chip, wherein the processing logic is configured to:
receive the LVO alert from the alert input, wherein the LVO alert is representative of a probability of an LVO event in a patient;
receiving a mapping coordinate from the GPS chip;
accessing a database of care centers proximate to the mapping coordinate;
selecting a preferred care center from the database based at least on: (1) a treatment capability associated with the preferred care center; and (2) a distance of the care centers to the mapping coordinate; and
transmitting, with the wireless radio, an LVO event notification to the preferred care center to initiate stroke medical care.

15. The device for early notification of LVO of claim 14, wherein the processing logic is further configured to:
receive a cerebrovascular image associated with the LVO alert, wherein transmitting the LVO event notification to the preferred care center with the wireless radio includes transmitting the cerebrovascular image.

16. The device for early notification of LVO of claim 14 further comprising:
a memory storing the database of care centers, wherein the processing logic is communicatively coupled to read the memory to access the database.

17. The device for early notification of LVO of claim 14, wherein accessing the database of care centers includes:
transmitting an access request to a database via the wireless radio, wherein the access request includes the mapping coordinate; and
receiving, in response to the access request, a database response that includes a plurality of care centers and resources and locations of the care centers, wherein the database response is received via the wireless radio.

18. The device for early notification of LVO of claim 14, wherein the LVO alert is received by the wireless radio, and wherein the wireless radio is configured to interface with at least one of a cellular network, a wireless local area network (WLAN), or a satellite communication system.

19. The device for early notification of LVO of claim 14, wherein the LVO alert includes an indicator of a subtype of LVO.

20. The device for early notification of LVO of claim 19, wherein the subtype of LVO is an ICA occlusion, an MCA occlusion, an M2 inferior occlusion, or an M2 superior occlusion.
